SEQUENCE LISTING (33 sequences)

SEQ ID NO: 1 - DNA, 21 bp, Mus musculus
  Target sequence of p85alpha
aagcagcaaccgaaacaaagc

SEQ ID NO: 2 - RNA, 21 nt, artificial sequence
  Synthetic sense strand of anti-p85alpha siRNA
gcagcaaccgaaacaaagcuu

SEQ ID NO: 3 - RNA, 21 nt, artificial sequence
  Synthetic antisense strand of anti-p85alpha siRNA
gcuuuguuucgguugcugcuu

SEQ ID NO: 4 - DNA, 21 bp, Mus musculus
  Target sequence of Akt1
aagatgacagcatggagtgtg

SEQ ID NO: 5 - RNA, 21 nt, artificial sequence
  Synthetic sense strand of anti-Akt1 siRNA
gaugacagcauggaguguguu

SEQ ID NO: 6 - RNA, 21 nt, artificial sequence
  Synthetic antisense strand of anti-Akt1 siRNA
cacacuccaugcugucaucuu

SEQ ID NO: 7 - DNA, 8 bp, artificial sequence
  T7 promoter primer sequence
cctgtctc

SEQ ID NO: 8 - DNA, 29 bp, artificial sequence
  Synthetic sense strand template for anti- p85alpha siRNA
aagctaggcgacctaataagccctgtctc

SEQ ID NO: 9 - DNA, 29 bp, artificial sequence
  Synthetic antisense strand template for anti- p85alpha siRNA
aagcttattaggtcgcctagccctgtctc

SEQ ID NO: 10 - DNA, 29 bp, artificial sequence
  Synthetic sense strand template for anti- p85alpha siRNA
aagggatgtgcgggtatattccctgtctc

SEQ ID NO: 11 - DNA, 29 bp, artificial sequence
  Synthetic antisense strand template for anti- p85alpha siRNA
aagaatatacccgcacatccccctgtctc

SEQ ID NO: 12 - DNA, 29 bp, artificial sequence
  Synthetic sense strand template for anti- p85alpha siRNA
aagctttgtttcggttgctgccctgtctc

SEQ ID NO: 13 - DNA, 29 bp, artificial sequence
  Synthetic antisense strand template for anti- p85alpha siRNA
aagcagcaaccgaaacaaagccctgtctc

SEQ ID NO: 14 - DNA, 29 bp, artificial sequence
  Synthetic sense strand template for anti-Akt1 siRNA
aaggccacgatgacttccttccctgtctc

SEQ ID NO: 15 - DNA, 29 bp, artificial sequence
  Synthetic antisense strand template for anti- Akt1 siRNA
aagaaggaagtcatcgtggcccctgtctc

SEQ ID NO: 16 - DNA, 29 bp, artificial sequence
  Synthetic sense strand template for anti-Akt1 siRNA
aagtgggtctggaaagagtaccctgtctc

SEQ ID NO: 17 - DNA, 29 bp, artificial sequence
  Synthetic antisense strand template for anti- Akt1 siRNA
aagtactctttccagacccaccctgtctc

SEQ ID NO: 18 - DNA, 29 bp, artificial sequence
  Synthetic sense strand template for anti-Akt1 siRNA
aacacactccatgctgtcatccctgtctc

SEQ ID NO: 19 - DNA, 29 bp, artificial sequence
  Synthetic antisense strand template for anti- Akt1 siRNA
aagatgacagcatggagtgtgcctgtctc

SEQ ID NO: 20 - DNA, 21 bp, artificial sequence
  Target sequence of pik3r1
aagcttattaggtcgcctagc

SEQ ID NO: 21 - RNA, 21 nt, artificial sequence
  Synthetic sense strand of anti-pik3r1 siRNA
gcuuauuaggucgccuagcuu

SEQ ID NO: 22 - RNA, 21 nt, artificial sequence
  Senthetic antisense strand of anti-pik3r1 siRNA
gcuaggcgaccuaauaagcuu

SEQ ID NO: 23 - DNA, 21 bp, artificial sequence
  Target sequence of pik3r1
aagaatatacccgcacatccc

SEQ ID NO: 24 - RNA, 21 nt, artificial sequence
  Synthetic sense strand of anti-pik3r1 siRNA
gaauauacccgcacaucccuu

SEQ ID NO: 25 - RNA, 21 nt, artificial sequence
  Synthetic antisense strand of anti-pik3r1 siRNA
gggaugugcggguauauucuu

SEQ ID NO: 26 - DNA, 21 bp, artificial sequence
  Target sequence of pik3r1
aagcagcaaccgaaacaaagc

SEQ ID NO: 27 - DNA, 21 bp, artificial sequence
  Target sequence of Akt1
aagaaggaagtcatcgtggcc

SEQ ID NO: 28 - RNA, 21 nt, artificial sequence
  Synthetic sense strand of anti-Akt1 siRNA
gaaggaagucaucguggccuu

SEQ ID NO: 29 - RNA, 21 nt, artificial sequence
  Synthetic antisense strand of anti-Akt1 siRNA
ggccacgaugacuuccuucuu

SEQ ID NO: 30 - DNA, 21 bp, artificial sequence
  Target sequence of Akt1
aagtactctttccagacccac

SEQ ID NO: 31 - RNA, 21 nt, artificial sequence
  Synthetic sense strand of anti-Akt1 siRNA
guacucuuuccagacccacuu

SEQ ID NO: 32 - RNA, 21 nt, artificial sequence
  Synthetic antisense strand of anti-Akt1 siRNA
gugggucuggaaagaguacuu

SEQ ID NO: 33 - DNA, 21 bp, artificial sequence
  Target sequence of Akt1
aagatgacagcatggagtgtg

(19) United States
(12) Patent Application Publication
(10) Pub. No.: US 2005/0272682 A1
(43) Pub. Date: Dec. 8, 2005

(54) SiRNA targeting PI3K signal transduction pathway and siRNA-based therapy

(76) Inventors: Bernard Mark Evers, Galveston, TX (US); Piotr Rychahou, Galveston, TX (US)

Correspondence Address: MUETING, RAASCH & GEBHARDT, P.A., P.O. BOX 581415, MINNEAPOLIS, MN, 55458, US

(21) Appl. No.: 11/085,962

(22) Filed: Mar. 22, 2005

Related U.S. Application Data
(60) Provisional application No. 60/555,275, filed on Mar. 22, 2004.

(51) Int. Cl. A61K 48/00; C07H 21/02

(52) U.S. Cl. 514/44; 536/23.1

(57) ABSTRACT

Small interfering RNAs (siRNAs) are provided specifically targeting a PI3K signal transduction pathway. Also provided are various siRNA-based therapies.

### CROSS-REFERENCE TO RELATED APPLICATION

This non-provisional application claims priority of U.S. provisional application No. 60/555,275, filed Mar. 22, 2004, now abandoned.

### GOVERNMENT FUNDING

This invention was made partly through the grants RO1CA104748, RO1DK48498, R37AG10885, PO1DK35608 and T32DK07639 from National Institute of Health. Therefore, the U.S. government has certain rights in this invention.

### BACKGROUND OF THE INVENTION

1. Field of the Invention

The present invention generally relates to molecular biology and medical treatment. More specifically, the present invention relates to small interfering RNAs (siRNAs) and siRNA-based therapy.

2. Description of the Related Art

Small interfering RNAs (siRNAs) are mediators of mRNA degradation, which are produced from long double-stranded RNA (dsRNA) by enzymatic cleavage in the cell. The siRNA are approximately twenty-one nucleotides in length and have a base-paired structure characterized by two-nucleotide 3′-overhangs. The siRNA can be rapidly synthesized corresponding to specific genes. The synthesized siRNA can be used to regulate expression and transactivation of the target genes.

Phosphoinositide 3-kinase (PI3K), a ubiquitous lipid kinase that is involved in receptor signal transduction pathway through tyrosine kinase receptors, is composed of a regulatory subunit (p85) and a 110-kDa catalytic subunit (p110). PI3K acts through its downstream effector protein kinase B (PKB, also named Akt) to regulate many cellular processes including cell survival, cell proliferation, vesicular trafficking, inflammation and apoptosis inhibition. Akt consists of three isoforms, with the Akt1 being the best described and more often reported isoform in cancers. When activated, PI3K phosphorylates phosphoinositides at its third carbon (Cantley, 2002; Wang, et al., 2002). Once phosphorylatyed, the phosphoinositides promote Akt activation by phosphorylation (Akt→Phospho-Akt (pAkt)), which results in the phosphorylation of many proteins that affect vesicular trafficking, actin rearrangement, cell growth, and cell survival (Cantley, 2002).

Chemokines and chemotactic factors (e.g., interleukin-8 (IL-8)) bind to G protein-coupled receptors on leukocytes. This receptor interaction triggers cell movement, phagocytosis, degranulation, and superoxide generation. Inhibition of the PI3K pathway blocks these responses. Inhibition of PI3K-Akt has been implicated in playing a pivotal role in cytokine-induced transcriptional activation of nuclear factor kappa B-(NF-κB-) and activating protein-1-(AP-1-) dependent gene expression and in activating apoptosis (Guha, et al., 2002). One recent study showed that in vitro, lipopolysaccharide (LPS)-stimulated monocytes caused an activation of the P13K pathway, which inactivated mitogen-activated protein kinase (MAPK) (extracellular signal-regulated kinase ½ (ERK½), p38, and c-Jun N-terminal kinase (JNK)) pathways and NF-κB by phosphorylation of various proteins, including glycogen synthase kinase-3 (GSK-3) and other protein kinases (Guha, et al., 2002). Several studies have shown that interleukin-8 (IL-8) expression is dependent on PI3K and NF-κB activation (Oswa, et al., 2002). Thus many proinflammatory proteins require PI3K activity to transmit inflammatory signals (Osawa, et al., 2002). It is known that inhibition of PI3K decreases neutrophil infiltration (Hirsch, et al., 2000).

PI3K regulatory subunits (p85) include p85α and its truncated splice variants p50α and p55α, as well as p85β and p55γ. The regulatory subunits p85α, p50α, and p55α are encoded by the pik3r1 gene; p85α is the most abundantly expressed regulatory isoform of PI3K, and p55α and p50α are two additional minor alternative splicing isoforms (Philp, et al., 2001; Jimenez, et al., 1998).

Catalytic subunit p110 of PI3K has four known Class I isoforms, i.e., p110α, p110β, p110γ and p110δ. Leukocytes express all these known isoforms. It is unclear which isoform is responsible for inflammation. Okkenhaug, et al. (2002) reported that mice expressing the catalytically inactive form of p110δ (i.e., p110δ knockout mice) developed mild inflammatory bowel disease in colorectal tissues characterized by mucosal hyperplasia, crypt abscesses and mixed leukocyte infiltration.

Inflammatory bowel disease is a group of diseases that affect the mucosal lining of the gastrointestinal tract. The incidence and prevalence of these diseases depend on the population in question, but overall, ulcerative colitis is more frequent than Crohn's disease. Combined prevalence of these diseases ranges from 90-190 cases per 100,000 people (Glickman, 1998). Patients affected by inflammatory bowel disease suffer from a variety of symptoms including abdominal pain and bloating, frequent diarrhea, rectal bleeding, and have an increased risk of colorectal cancer. The exact cause of the inflammation is unknown. Evidence currently available, however, suggests an aggregate affect of genetic and environmental factors that lead to sustained activation of the mucosal immune response. A decreased ratio of interleukin-1 (IL-1) receptor to its ligand, IL-1, correlates to the severity of ulcerative colitis and Crohn's disease patients (Evers, 2001). One study shows that increased mucosal inflammation in inflammatory bowel disease in children is associated with a decrease in the “normal” effective IL-1ra/IL-1 beta ratio in which IL-1ra predominates. Additionally, IL-1β and IL-6 cytokines are known to be elevated in rectal tissues of ulcerative colitis patients (Grottrup-Wolfers, et al., 1996).

Colorectal cancer is another type of colonic mucosal disease. There is a growing body of evidence to support the notion that the activation of PI3K/Akt is associated with colorectal carcinoma and can convert differentiated human gastric or colonic carcinoma cells to a less differentiated and more malignant phenotype (Semba, et al., 2002). Akt is overexpressed in a number of cancers, including colon, pancreatic, ovarian and some steroid hormone insensitive breast cancers (Roy, et al., 2002; Asano, et al., 2004; Page, et al., 2000). Moreover, it has been reported that Akt phosphorylation in human colon carcinomas correlates with cell proliferation and apoptosis inhibition, as well as with different clinicopathologic parameters such as invasion grade, vessel infiltration, metastasis to lymph nodes, and tumor stage (Khaleghpour, et al., 2004; Itoh, et al., 2002; Wang, et al., 2002).

Current therapies for treating colonic mucosal diseases are limited to systemic administration or direct application of certain chemicals. Systemic administration limits the maximum dose of all chemicals. Systemic circulation reduces the bioavailability of treatments to specific target tissues due to normal chemical breakdown observed in the liver and vascular beds. To compensate the loss of the chemicals in non-target tissues, systemic administration would require a higher dose of the chemicals. Side effects of chemical medications, however, are dose dependant. With the increased dose of the chemicals administered to the patient to reach the threshold target tissue bioavailability, side effects of the medications may be a significant factor in morbidity and mortality. In fact, therapies currently available are not effective for treating inflammation within the colon or marginally effective in preventing the inflammation from progressing.

Synthesized siRNA targeting specific genes are now broadly available for the analyses of the gene function in cultured mammalian cells. Similar to antisense oligonucleotide technology, the use of siRNA also holds great promise for the application of gene-specific therapies. The delivery of siRNA to the target sites of therapy, however, remains problematic. This is especially true for their delivery to primary cells as such cells often do not tolerate treatment with liposome transfection reagents. Chemical modification of siRNA such as changing the lipophilicity of the molecule may be considered, for example, phosphorothioate modifications present in antisense oligodeoxynucleotides, or the attachment of lipophilic residues at the 3′-termini of the siRNA. Delivery of siRNA into organisms might be achieved with methods previously developed for the application of antisense oligonucleotides or nuclease-resistant ribozymes. Such methods usually include a step of injecting naked or liposome-encapsulated molecules. These traditional delivery methods are not specifically targeted at tissues that require the therapy, and accordingly, the efficacy of the treatment is not promising.

Therefore, there is a need for an effective method for treating colonic mucosal disease and other types of inflammatory diseases.

### SUMMARY OF THE INVENTION

The present invention is advantageously directed to small interfering RNAs (siRNAs) specifically targeting a PI3K signal transduction pathway. The present invention is also advantageously directed to various siRNA-based therapies.

In one embodiment of the present invention, there is provided small interfering RNA (siRNA) which target a phosphoinositide 3-kinase (PI3K) signal transduction pathway. The siRNA is characterized as being directed against p85α of the PI3K signal transduction pathway or against Akt of the PI3K signal transduction pathway. The anti-p85α siRNA are double-stranded, with a first strand comprising a sequence selected from the group consisting of SEQ ID NO:2, SEQ ID NO:21, and SEQ ID NO:24, and a second strand comprising a sequence selected from the group consisting of SEQ ID NO:3, SEQ ID NO:22, and SEQ ID NO:25. The anti-Akt siRNA are double-stranded, with a first strand comprising a sequence selected from the group consisting of SEQ ID NO:5, SEQ ID NO:28, and SEQ ID NO:31, and a second strand comprising a sequence selected from the group consisting of SEQ ID NO:6, SEQ ID NO:29, and SEQ ID NO:32.

In another embodiment of the present invention, there is provided a method for treating an individual suffering from an inflammatory disease. This method advantageously includes administering to the individual with a selective inhibitor targeting a specific component of inflammatory cascade propagation through a localized delivery.

In still another embodiment of the present invention, there is provided a method for treating an individual suffering from an inflammatory disease of colonic mucosa. This method advantageously includes administering to the individual with a selective inhibitor targeting a PI3K signal transduction pathway through a localized delivery.

In still yet another embodiment of the present invention, there is provided a method for treating an individual having a cancer, and more particularly, a chemoresistant cancer. This method advantageously includes co-administering to the individual with a chemotherapeutic drug and a selective inhibitor targeting a PI3K signal transduction pathway.

In still yet another embodiment of the present invention, there is provided a method for increasing efficacy of chemotherapy in a cancer treatment. This method advantageously includes co-administering to an individual in need of such cancer treatment with a chemotherapeutic drug and a selective inhibitor targeting a PI3K signal transduction pathway.

In still yet another embodiment of the present invention, there is provided a pharmaceutical composition comprising siRNA targeting a PI3K signal transduction pathway and a pharmaceutically acceptable carrier.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

The application file contains at least one drawing executed in color. Copies of this patent application publication with color drawing(s) will be provided by the Office upon request and payment of the necessary fee.

Features of the present invention as well as a preferred mode of use, further objectives, and advantages thereof, will best be understood by reference to the following detailed description of an illustrative embodiment when read in conjunction with the accompanying drawings, wherein:

FIG. 1 is a series of histology sectional views of colonic tissue from normal mice (control, panel A), from dextran sulfate sodium (DSS)-induced colitis mice (panel B), from wortmannin treatment only mice (without DSS exposure) (panel C) and DSS-induced colitis mice post wortmannin treatment (panel D).

FIG. 2 is a bar graph illustrating measurement of MPO activity, cytokine tissue levels (IL-1β, IL-6, MIP-2, TNFα) and p65 NF-κB activity in mice from different experimental groups.

FIG. 3 is a line graph illustrating disease activity index (DAI) (panel A) and weight changes (%) (panel B) of mice in different experimental groups.

FIG. 4 is a series of histology sectional views of proximal colon (panel A), distal colon (panel B), liver (panel C) and spleen (panel D) of mice treated with Cy-3-labeled siRNA intrarectally.

FIG. 5 is a photograph illustrating colons from mice in different experimental groups.

FIG. 6 is a series of histology sectional views of colons from mice treated with scram siRNA (panel A), DSS+scram siRNA (panel B), DSS+p85α siRNA (panel C) and DSS+Akt1 siRNA (panel D).

FIG. 7 is an electrophoresis gel illustrating expression levels of pAkt, p85α, Akt, and p110δ in mice from different experimental groups as measured by Western blotting.

FIG. 8 is a line graph illustrating disease activity index (DAI) (panel A) and weight changes (%) (panel B) of mice in different experimental groups.

FIG. 9 is a bar graph illustrating measurement of MPO activity, cytokine tissue levels (IL-1β, IL-6, MIP-2, TNFα) and p65 NF-κB activity in mice treated with Akt1 siRNA.

FIG. 10 is a bar graph illustrating measurement of MPO activity, cytokine tissue levels (IL-1β, IL-6, MIP-2, TNFα) and p65 NF-κB activity in mice treated with p85α siRNA.

FIG. 11 is a photograph illustrating colons from mice in different experimental groups.

FIG. 12 is a series of histology sectional views of colons from mice treated with scram siRNA (panel A), trinitrobenzene sulfonic acid (TNBS)+scram siRNA (panel B), TNBS+Akt1 siRNA (panel C) and TNBS+p85α siRNA (panel D).

FIG. 13 is a bar graph illustrating measurement of MPO activity, cytokine tissue levels (IL-1β, IL-6, MIP-2, TNFα) and p65 NF-κB activity in mice from different experimental groups.

FIG. 14 is an electrophoresis gel illustrating expression levels of pAkt, p85α, Akt, and p110δ in mice from different experimental groups as measured by Western blotting.

FIG. 15A is an electrophoresis gel illustrating inhibition of Akt1 expression in KM20 cells treated with siRNA directed against Akt1 as measured by Western blotting.

FIG. 15B is an electrophoresis gel illustrating inhibition of p85α expression in KM20 cells treated with siRNA directed against pik3r1 as measured by Western blotting.

FIG. 15C is a panel of three confocal microscopic views illustrating pAkt expression in KM20 cells transfected with scrambled (control), pik3r1, or Akt1 siRNA sequences and stained with pAkt.

FIG. 16A is a line graph illustrating inhibition of proliferation of KM20 cells by siRNA directed against pik3r1 or Akt1.

FIG. 16B is a line graph illustrating inhibition of proliferation of KM12C cells by siRNA directed against pik3r1 or Akt1.

FIG. 16C is a bar graph illustrating DNA fragmentation in KM20 and KM12C cells transfected with siRNA directed against either pik3r1, Akt1 individually or in combination or scrambled (control) sequence.

FIG. 17A is a pair of electrophoresis gels illustrating mitochondrial apoptotic pathway activation in KM20 or KM12C cells treated with either pik3r1, Akt1 or scrambled (control) siRNA sequences as measured by Western blotting.

FIG. 17B is a pair of electrophoresis gels illustrating mitochondrial apoptotic pathway activation in KM20 or KM12C cells treated with either pik3r1, Akt1 or scrambled (control) siRNA sequences as measured by Western blotting.

FIG. 17C is a series of electrophoresis gels illustrating mitochondrial apoptotic pathway activation in KM20 or KM12C cells treated with either pik3r1, Akt1 or scrambled (control) siRNA sequences as measured by ribonuclease (RNAse) protection assays.

FIG. 18 is a line graph illustrating effects of pik3r1 or Akt1 siRNA on KM20 tumor growth in vivo.

FIG. 19 is an electrophoresis gel illustrating reduced expression of pAkt and anti-apoptotic proteins in tumor xenografts of mice treated with Akt1 siRNA (panel A) or pik3r1 siRNA (panel B) as measured by Western blotting.

FIG. 20 is an electrophoresis gel illustrating apoptotic pathway activation in tumor xenografts of mice treated with Akt1 siRNA (panel A) and pik3r1 siRNA (panel B) as measured by Western blotting.

FIG. 21A is an electrophoresis gel illustrating increased expression of death receptor DR4 and DR5 in KM20 cells transfected with pik3r1 or Akt1 siRNA as measured by ribonuclease protection assay.

FIG. 21B is an electrophoresis gel illustrating increased expression of death receptor DR4 and DR5 in KM20 cells transfected with pik3r1 or Akt1 siRNA as measured by Western blotting.

FIG. 22A is a pair of bar graphs illustrating induction of TNF-related apoptosis-inducing ligand (TRAIL)-mediated apoptosis in KM20 or KM12C cells transfected with pik3r1 siRNA, Akt1 siRNA or scrambled control.

FIG. 22B is a bar graph illustrating induction of TRAIL-mediated apoptosis in KM20 cells treated with the caspase-3 inhibitor prior to the treatment of TRAIL and pik3r1 or Akt1 siRNA.

FIG. 23 is an electrophoresis gel illustrating Caspase activation following pik3r1 or Akt1 transfection and TRAIL treatment in KM20 cells as measured by Western blotting.

FIG. 24 is a pair of photographs illustrating blockage of edema, hemorrhage and pleural effusion by p85α siRNA during severe acute pancreatitis (panel B) compared to control siRNA treatment (panel A).

FIG. 25 is a graph illustrating reduction of mortality of severe acute pancreatitis by p85α siRNA treatment.

### DETAILED DESCRIPTION

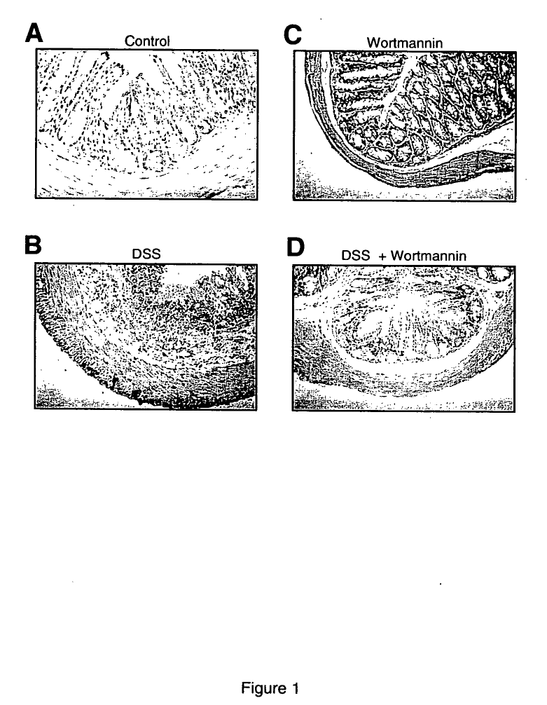

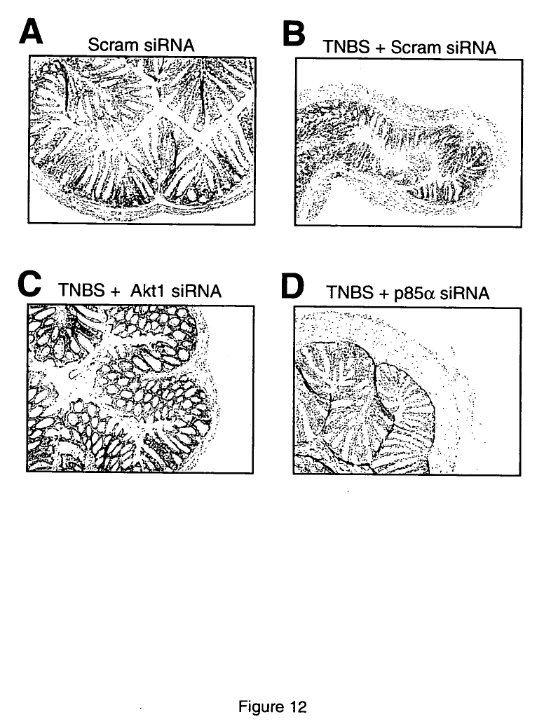

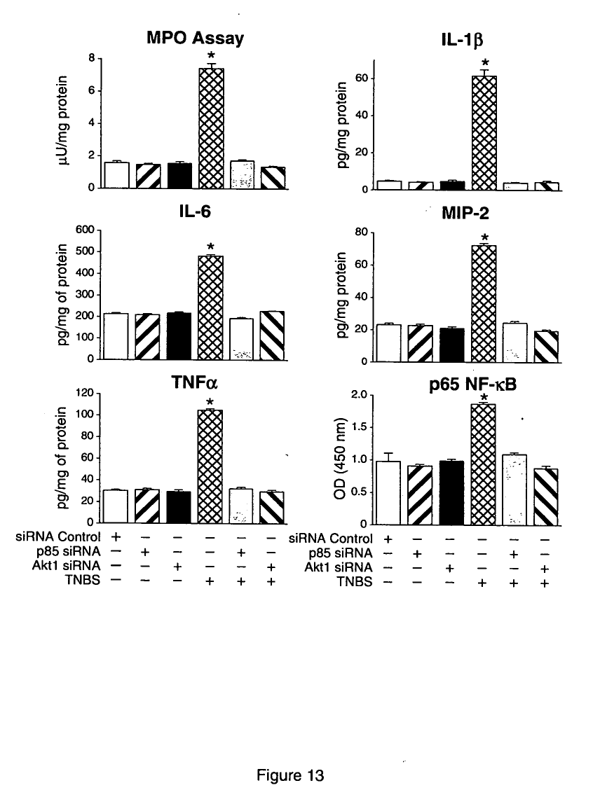

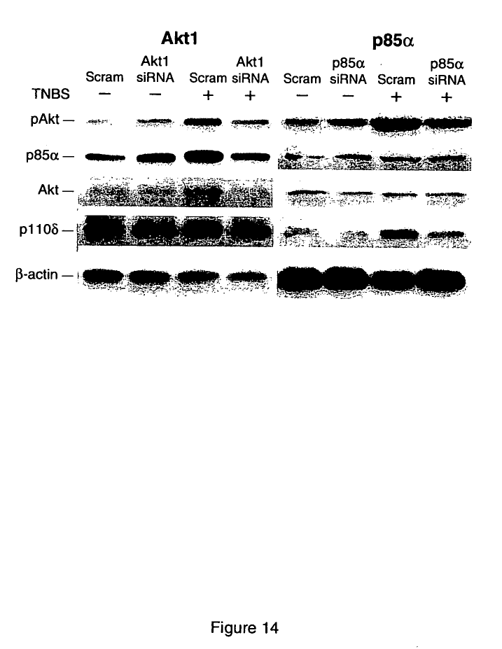

As illustrated in FIGS. 1-25 and as further described herein, the present invention advantageously provides small interfering RNAs (siRNAs) specifically targeting a phosphoinositol 3-kinase (PI3K) signal transduction pathway. Also advantageously provided are various siRNA-based therapies.

Small interfering RNAs (siRNAs) were synthesized corresponding to components of the PI3K signal transduction pathway, which include the p85α regulatory enzyme of PI3K and the downstream effector gene Akt. Anti-p85α siRNA (e.g., pik3r1 siRNA) serves to directly inhibit the function of the PI3K enzyme within the colonic mucosa. Anti-Akt siRNA (e.g., Akt1 siRNA) serves to directly inhibit downstream signaling of the PI3K enzyme but does not affect the PI3K enzyme itself.

Anti-p85α siRNA and anti-Akt1 siRNA were used in the present study to treat a murine model of colitis. In detail, a suspension of anti-p85α siRNA or anti-Akt1 siRNA was given in two different routes of administration. One form of administration was through a rectal enema. In this method, a small plastic tube was inserted via the anus of the mice and approximately 8 cm into the colon. A solution of suspended anti-p85α siRNA or anti-Akt1 siRNA is infused into the tube at a daily dose of 100 μl in volume for three days over the course of the colitis model (typically 7 days). The second form of administration was through a systemic route, e.g., intraperitoneal (IP) injection. A solution of suspended anti-p85α siRNA or anti-Akt1 siRNA was injected on the ventral side of the mice once daily at a daily dose of 100 μl in volume for three days of the 7-day-course of the colitis model.

As a result of the siRNA treatment, inhibition of the PI3K/Akt pathway by the intrarectal instillation of small interfering RNAs (siRNAs) directed to either p85α or Akt1 markedly attenuates the inflammatory changes in the colonic mucosa of mice given either dextran sulfate sodium (DSS) or trinitrobenzene sulfonic acid (TNBS) as demonstrated by decreased disease activity index (DAI), neutrophils sequestration, cytokine tissue levels and NF-κB activity. DSS treatment is characterized by a destruction of the colonic epithelial layer and a polymorphonuclear cell infiltrate involving the mucosa and submucosa, while TNBS treatment results in inflammation marked by a transmural cellular infiltration with T cells and macrophages in a pattern like that described in interleukin-2 knockout mice and Crohn disease. Consistent with these results, Cy-3-labeled siRNA was detected in the colonic wall as well as systemically in the spleen and liver suggesting enhanced delivery into the mucosa following local instillation. This indicates that selective targeting of the PI3K/Akt pathway by intrarectal siRNA may provide a novel method to attenuate the acute inflammatory changes associated with inflammatory bowel disease.

In the present study, target tissues are directly treated with siRNA through rectal administration. Such direct delivery requires minimal doses of the siRNA as systemic breakdown is virtually eliminated. Since the siRNA is directed to a specific signal transduction pathway (i.e., PI3K), the direct siRNA therapy leads to a decrease in inflammation within the tissue at a molecular level and treats the disease with minimal side effects, as other vital cellular systems are not affected.

Similarly, anti-p85α siRNA was also used to treat a murine model of severe acute pancreatitis. The results showed that anti-p85α siRNA reduced the development of acute pancreatitis and decreased the mortality.

Targeted inhibition of PI3K/Akt using siRNA directed to either the p85α regulatory subunit (pik3r1) or Akt1 is also shown to alter colorectal cancer growth and enhance apoptosis in the present study. Transfection of KM20 or KM12C colorectal cancer cell lines with either pik3r1 or Akt1 siRNA inhibited cell proliferation and rendered the cells sensitive to apoptosis by increased cytochrome C release and caspase-9 and caspase-3 activation. Furthermore, transfection with pik3r1 or Akt1 siRNA significantly decreased expression levels of the anti-apoptotic proteins Bcl-2 and Bcl-xL and increased expression of the proapoptotic protein Bax. Using a nude mouse xenograft model, electroporation of siRNA directed to either pik3r1 or Akt1 is shown to decrease KM20 tumor growth over a time course. Similar to the in vitro findings, decreased expression of Mcl-1 and Bcl-xL proteins and increased expression of Bax was noted in the xenografts transfected with the siRNA to either pik3r1 or Akt1 compared with transfection of scrambled control sequence. In addition, increased activation of caspase-8 and caspase-3, cleavage of PARP and increased cytochrome C was noted in the siRNA-treated tumors. The results demonstrate that transient siRNA-mediated knockdown of the p85α regulatory subunit or Akt1 leads to inhibition of colorectal cancer cell growth through the induction of the intrinsic apoptotic pathway. Specific inhibition of PI3K/Akt pathway components may provide novel adjuvant treatment for selected colorectal cancers.

The present study also demonstrated that combination treatment with p85α or Akt1 siRNA and TNF-related apoptosis-inducing ligand (TRAIL) increased apoptosis in KM20 and KM12C cells compared with TRAIL alone. The results were further corroborated by complete inhibition of apoptosis by Z-DEVD-fmk, a caspase-3 inhibitor. Furthermore, siRNA-mediated PI3K pathway inhibition resulted in increased expression of the DR4 and DR5 TRAIL receptors. This indicates that inhibition of PI3K/Akt by RNA interference sensitizes resistant colorectal cancer cells to TRAIL-induced cell death through the induction of TRAIL receptors and activation of caspase-3 and caspase-8. Agents which selectively target the PI3K/Akt pathway may enhance the effects of chemotherapeutic agents and provide novel adjuvant treatment for selected colorectal cancers.

In the present invention, the term “inflammatory disease” shall refer to any pathological condition that is either derived from inflammation or not derived from inflammation but has a link to inflammation. For example, a cancer such as a colorectal cancer is considered as an inflammatory disease in the present invention. Although they are not known to be derived from inflammation, most colorectal cancers are linked to inflammation since patients with inflammatory bowel disease (e.g., ulcerative colitis and Crohns disease) are much more likely to develop colorectal cancers than the normal population. Other examples of inflammatory diseases covered in the present invention include inflammatory bowel disease, pancreatitis, colonic dysplasia, enterocolitis, arthritis, an autoimmune disease, a chronic inflammatory state associated with organ transplantations and a chronic inflammatory state associated with infection, a toxin or allergy.

In one embodiment of the present invention, there is provided small interfering RNA (siRNA) targeting a phosphoinositide 3-kinase (PI3K) signal transduction pathway. Advantageously, the siRNA is characterized as being directed against p85α of the PI3K signal transduction pathway and being double-stranded, with a first strand comprising a sequence selected from the group consisting of SEQ ID NO:2, SEQ ID NO:21, and SEQ ID NO:24, and a second strand comprising a sequence selected from the group consisting of SEQ ID NO:3, SEQ ID NO:22, and SEQ ID NO:25. Alternatively, the siRNA is characterized as being directed against Akt of the PI3K signal transduction pathway and being double-stranded, with a first strand comprising a sequence selected from the group consisting of SEQ ID NO:5, SEQ ID NO:28, and SEQ ID NO:31, and a second strand comprising a sequence selected from the group consisting of SEQ ID NO:6, SEQ ID NO:29, and SEQ ID NO:32.

In another embodiment of the present invention, there is provided a method for treating an individual suffering from an inflammatory disease. This method advantageously includes administering to the individual with a selective inhibitor targeting a specific component of inflammatory cascade propagation through a localized delivery. The selective inhibitor advantageously comprises small interfering RNAs. Further advantageously, the inflammatory cascade propagation comprises phosphoinositide 3-kinase (PI3K) signal transduction pathway, and the selective inhibitor comprises small interfering RNAs (siRNAs) directed against components of PI3K signal transduction pathway.

The selective inhibitor advantageously comprises siRNA directed against p85α of the PI3K signal transduction pathway. The siRNA advantageously are double-stranded, with a first strand comprising a sequence selected from the group consisting of SEQ ID NO:2, SEQ ID NO:21, and SEQ ID NO:24, and a second strand comprising a sequence selected from the group consisting of SEQ ID NO:3, SEQ ID NO:22, and SEQ ID NO:25. Alternatively, selective inhibitor advantageously comprises siRNA directed against Akt of the PI3K signal transduction pathway. The siRNA advantageously are double-stranded, with a first strand comprising a sequence selected from the group consisting of SEQ ID NO:5, SEQ ID NO:28, and SEQ ID NO:31, and a second strand comprising a sequence selected from the group consisting of SEQ ID NO:6, SEQ ID NO:29, and SEQ ID NO:32.

Inflammatory diseases as referred herein comprise conditions that have associated inflammation which may be involved in the pathology. Representative examples of inflammatory diseases include cancers, inflammatory bowel disease, pancreatitis, colonic dysplasia, enterocolitis, arthritis, autoimmune diseases (e.g., Hashimoto's thyroiditis, lupus), chronic inflammatory states associated with organ transplantations, and chronic inflammatory states associated with infection, a toxin or allergy.

This method further advantageously comprises the step of directly delivering the selective inhibitor to a diseased tissue of the individual. The diseased tissue can be further exposed to electric pulses.

In still another embodiment of the present invention, there is provided a method for treating an individual suffering from an inflammatory disease of colonic mucosa. This method advantageously includes administering to the individual with a selective inhibitor targeting a phosphoinositide 3-kinase (Pl3K) signal transduction pathway through a localized delivery. Advantageously, the selective inhibitor comprises small interfering RNAs (siRNAs). Further advantageously, the selective inhibitor comprises siRNA directed against p85α of the PI3K signal transduction pathway. The siRNA advantageously are double-stranded, with a first strand comprising a sequence selected from the group consisting of SEQ ID NO:2, SEQ ID NO:21, and SEQ ID NO:24, and a second strand comprising a sequence selected from the group consisting of SEQ ID NO:3, SEQ ID NO:22, and SEQ ID NO:25. Alternatively, selective inhibitor advantageously comprises siRNA directed against Akt of the PI3K signal transduction pathway. The siRNA advantageously are double-stranded, with a first strand comprising a sequence selected from the group consisting of SEQ ID NO:5, SEQ ID NO:28, and SEQ ID NO:31, and a second strand comprising a sequence selected from the group consisting of SEQ ID NO:6, SEQ ID NO:29, and SEQ ID NO:32.

Representative examples of the inflammatory disease of colonic mucosa include colorectal cancers, inflammatory bowel disease, colonic dysplasia, and enterocolitis.

This method further advantageously comprises the step of directly delivering the selective inhibitor to a diseased tissue of the individual through a rectal enema. The diseased tissue can be further exposed to electric pulses.

In still yet another embodiment of the present invention, there is provided a method for treating an individual having a cancer, and more particularly, a chemoresistant cancer. This method advantageously includes co-administering to the individual with a chemotherapeutic drug and a selective inhibitor targeting a PI3K signal transduction pathway. Advantageously, the selective inhibitor comprises siRNA directed against p85α of the PI3K signal transduction pathway, which advantageously are double-stranded, with a first strand comprising a sequence selected from the group consisting of SEQ ID NO:2, SEQ ID NO:21, and SEQ ID NO:24, and a second strand comprising a sequence selected from the group consisting of SEQ ID NO:3, SEQ ID NO:22, and SEQ ID NO:25. Alternatively, the selective inhibitor advantageously comprises siRNA directed against Akt of the PI3K signal transduction pathway, which advantageously are double-stranded, with a first strand comprising a sequence selected from the group consisting of SEQ ID NO:5, SEQ ID NO:28, and SEQ ID NO:31, and a second strand comprising a sequence selected from the group consisting of SEQ ID NO:6, SEQ ID NO:29, and SEQ ID NO:32. Representative examples of chemotherapeutic drugs include apoptosis-inducing agents, a NF-κB inhibitors, growth factor inhibitors, cell proliferation inhibitors and inhibitors of MDR gene expression. An example of the apoptosis-inducing agent is a TNF-related apoptosis inducing ligand (TRAIL).

In still yet another embodiment of the present invention, there is provided a method for increasing efficacy of chemotherapy in a cancer treatment. This method advantageously includes co-administering to an individual in need of such cancer treatment with a chemotherapeutic drug and a selective inhibitor targeting a PI3K signal transduction pathway.

A pharmaceutical composition may be further advantageously provided comprising isolated and purified siRNA targeting a phosphoinositide 3-kinase (Pl3K) signal transduction pathway and a pharmaceutically acceptable carrier.

Further more, a system of administering siRNA-based therapy to an in-need individual might be advantageously provided. In this system, isolated and purified siRNA are administered to the individual suffering from an inflammatory disease via intraperitoneal (IP) injection and rectal enema.

The following examples are given for the purpose of illustrating various embodiments of the invention and are not meant to limit the present invention in any fashion.

### EXAMPLE 1

Murine Model of Inflammatory Bowel Disease

60 male C57/bl6 mice aged 8 weeks and weighed around 20 g were administered with 5% (w/v) dextran sulfate sodium (DSS) in drinking water for 7 days. DSS used was 5000 MW sulfated polysaccharide (Sigma Chemical Co., St. Louis, Mo.). It is believed that DSS has direct cytotoxic effects on epithelial cells, immune cell function interference, and luminal microflora. DSS-induced inflammatory bowel disease (IBD) was characterized by epithelial damage, mucosal inflammatory infiltrates, and luminal ulceration similar to ulcerative colitis (see FIG. 1, panels A and B).

A validated clinical disease activity index (DAI) (Murano, et al., 2000; Murthy, et al., 1993) ranging from a score of 0 to 4 was calculated by an investigator blinded to the protocol using the following parameters: stool consistency, presence of fecal blood, and changes in body weight (Murano, et al., 2000). The stool consistency was assessed using the following four point scale: 0, normal; 1, soft; 2, very soft but formed; and 3, liquid. The intensity of the hemoccult paper test was scored by the following scale: 0, negative; 1, faintly blue; 2, moderately blue; 3, dark blue; and 4, blood visible.

### EXAMPLE 2

Experimental Design

To determine whether the colonic inflammation noted in the murine model of inflammatory bowel disease was mediated through the PI3K/Akt pathway, experiments were designed to determine whether pAkt as a measure of PI3K activity increased in intestinal tissue of the murine model or conversely, whether inhibition of PI3K pathway decreased inflammation and cytokine production in intestinal tissue of the murine model.

Wortmannin, a natural and non-selective inhibitor of PI3K pathway, was given to the DSS-induced colitis mice. Mice were separated into 4 groups: i) vehicle control (PBS in 10% DMSO, and 5% ETOH); ii) wortmannin alone administered by oral gavage daily at a dose of 1.5 mg/kg (total volume of 100 μl), starting on the 2^{nd} day of DSS exposure and continued to day 7 of exposure; iii) DSS (5 %)+vehicle; and iv) DSS+wortmannin. Mice were weighed daily and sacrificed on day 7. Large intestine was removed at sacrifice and either snap-frozen for protein extraction or placed in formalin for histologic analysis.

Histology sections of large intestine were stained with H&E to evaluate degree of inflammatory changes in the mice before and after the wortmannin treatment. Neutrophil sequestration was assessed through MPO assay. Once the protein was extracted, Western blot was performed to determine expression levels of PI3K isozymes (p85α and p110δ), and Akt, phosphorylated (i.e., activated) Akt (pAkt). β-actin was used as a loading control. Levels of cytokines (e.g., IL-1β, IL-6, MIP-2 and TNFα) and p65 NF-κB concentration were also assessed in colonic tissues through cytokine ELISA.

### EXAMPLE 3

Attenuation of Mucosal Inflammation Associated with DSS-Induced Colitis with Wortmannin Treatment

After DSS exposure, the murine model of IBD showed increased leukocyte infiltration, submucosal edema, and extensive mucosal damage (FIG. 1, panels A and B). Treatment with wortmannin reduced leukocyte infiltration and submucosal edema but mucosal damage was still evident (FIG. 1, panels C and D). It is demonstrated that PI3K inhibition by wortmannin decreased submucosal edema, leukocyte infiltration, and mucosal disruption extensively in DSS-exposed groups. That is, PI3K inhibition decreased degree of inflammation significantly.

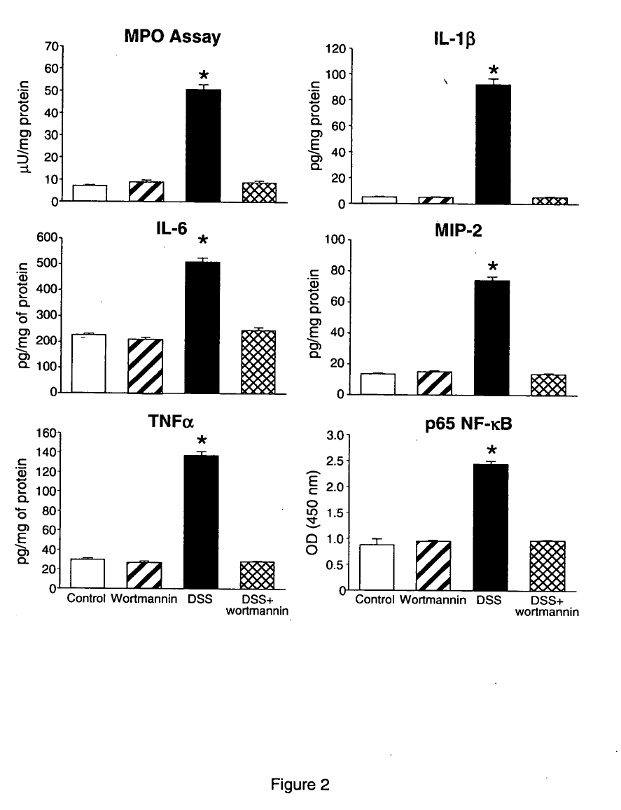

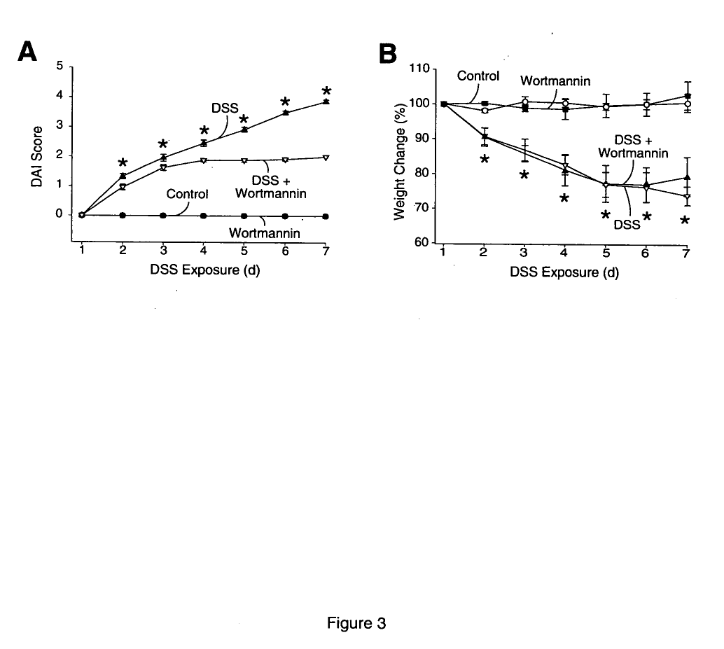

Mice given DSS exhibited a severe course of colitis with weight loss, bloody diarrhea and lethargy and a mean DAI of 4 by day 7 (FIG. 2, panel A). Treatment with wortmannin decreased the mean DAI score to 2 but no improvement was noted in the weight loss associated with DSS (FIG. 2, panel B). As further evidence of the amelioration in inflammation noted with wortmannin, MPO activity (a measure of leukocyte infiltration), tissue levels of cytokines (e.g., IL-1β, IL-6, MIP-2 and TNFα) and p65 NF-κB activity were measured (FIG. 3). DSS alone increased MPO activity, mucosal cytokine levels and p65 NF-κB activity. In contrast, treatment with wortmannin significantly reduced these parameters of inflammation. Wortmannin alone had no significant effect on DAI, histology or mucosal cytokine levels compared with control mice given vehicle gavage. Together, these results suggest that PI3K inhibition can attenuate the inflammatory changes associated with DSS-induced colitis.

### EXAMPLE 4

Selective PI3K Inhibitors

The above experiments using a natural and non-selective inhibitor of PI3K pathway (i.e., wortmannin) demonstrated that inhibition of PI3K attenuated the inflammation in colitis mice. Systemic administration of wortmannin effectively attenuates many of the inflammatory changes associated with DSS-induced colitis; however, wortmannin has short half-life (Jones, et al., 1999) and can exhibit nonspecific effects and is toxic with chronic administrations (Boehle, et al., 2002). It is proposed that selective inhibitors of PI3K isoforms could also attenuate inflammation, which might provide a novel treatment of inflammatory diseases including inflammatory bowel disease.

Small interfering RNAs (siRNAs) were designed to target components of murine PI3K signal transduction pathway, which include the p85α regulatory enzyme of PI3K and the downstream effector gene Akt. Anti-p85α siRNA targets regions of Mus musculus PI3K regulatory subunit p85α mRNA (5′-aagcagcaaccgaaacaaagc-3′, SEQ ID NO:1) and comprises a sequence as shown in SEQ ID NO:2 (gcagcaaccgaaacaaagcuu, sense strand) or SEQ ID NO:3 (gcuuuguuucgguugcugcuu, antisense strand). Anti-Akt siRNA targets regions of Mus musculus Akt1 mRNA (5′-aagatgacagcatggagtgtg-3′, SEQ ID NO:4) and comprises a sequence as shown in SEQ ID NO:5 (gaugacagcauggaguguguu, sense strand) or SEQ ID NO:6 (cacacuccaugcugucaucuu, antisense strand). The scrambled siCONTROL and custom siSTABLE (a chemically-modified siRNA) siRNA duplexes were synthesized at Dharmacon Research, Inc. (Lafayette, Colo.). Scrambled siCONTROL (control siRNA) was labeled with Silencer™ siRNA Labeling Kit—Cy™3 (Ambion Inc., Austin, Tex.) according to manufacturer instructions. 1,2 Dioleoyl-3-trimethylammonium-propane (DOTAP) was purchased from Roche Diagnostics.

### EXAMPLE 5

Experimental Design Using Selective PI3K Inhibitors

Inflammatory bowel disease (IBD) was induced in 2-month-old C57/bl6 mice using a 5% dextran sulfate sodium (DSS) solution in drinking water. siRNA directed to either p85α or Akt1 or siCONTROL was given on days 2, 3 and 4 of DSS exposure. Due to the short half-life of siRNA in vivo, siSTABLE was used which renders siRNA more resistant to degradation resulting in a half-life of 5 days. For each mouse, 5 μg of siRNA in 25 μl of transfection buffer was transferred into a sterile Eppendorf tube. In a separate sterile polystyrene tube 20 μg of DOTAP was mixed with 70 μl transfection buffer and then the siRNA mixture was transferred to the polystyrene tube containing the DOTAP and then incubated at room temperature for 30 min. Around 100 μl of the mixture was administered via the anus, by slow instillation into the lumen of the colon using a 20-gauge catheter and a 1-ml syringe. After treatment, mice were suspended head down for 30 sec. The colonic enema was performed on the second day of DSS exposure and repeated 24 and 48 h later. Cy-3 labeled siRNA were prepared for the treatment and administered as described above; mice were sacrificed at 4 and 24 hours after treatment and spleen, liver, pancreas and colon samples organs were taken and then frozen in liquid N₂. Sections (6 μm) were cut with a cryostat microtome, thaw-mounted on a glass slide (cleaned with ethanol) and analyzed by fluorescent light microscopy (excitation λ=570 ηm).

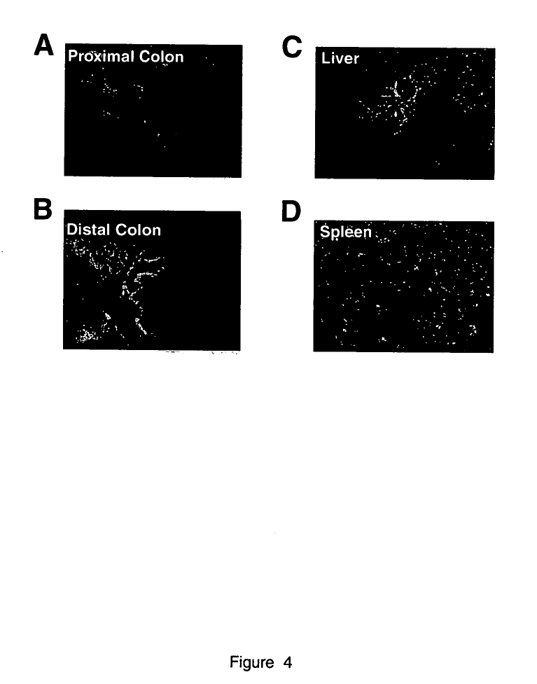

Given the relatively poor tissue uptake of siRNA by the intravenous or intraperitoneal routes (Sorensen, et al., 2003), intrarectal administration of siRNA was studied. As an initial feasibility study, trypan blue was administered intrarectally in mice; delivery was noted throughout the colon and base of the cecum. To assess the extent of tissue penetration and delivery of intrarectal siRNA, Cy-3-labeled siRNA was infused intrarectally in mice. Fluorescent light microscopy demonstrated Cy-3 staining in all layers of the proximal and distal colon wall at 4 h after administration (FIG. 4, panels A and B). At 24 h after administration, siRNA was noted only at the outer layer (i.e., serosa) of the colon (data not shown). In addition, siRNA was noted in the liver and spleen (FIG. 4, panels C and D) and pancreas (data not shown) at 24 h after intrarectal siRNA delivery.

### EXAMPLE 6

Protein Analysis, MPO Activity, Cytokine and NF-κB ELISA Assay and Morphology Analysis

Western immunoblot analyses were performed as described previously (Wang, et al., 2002). Individual colon samples were homogenized using a Polytron power homogenizer in cell lysis buffer at 4° C. for 30 min. Lysates were clarified by centrifugation (8,000 g for 20 min at 4° C.), and sonicated for 30-45 sec on ice. Protein concentrations were determined using the method of Bradford (1976). Total protein (100 μg) was resolved on a 10% polyacrylamide gel and transferred to Immobilon-P nylon membranes as described previously (Wang, et al., 2002). Filters were incubated overnight at 4° C. in blocking solution followed by a 1-h incubation with primary antibodies. Filters were washed three times in blocking solution and incubated with horseradish peroxidase-conjugated secondary antibody for 1 h. After three additional washes, the immune complexes were visualized by enhanced chemiluminescence (ECL) from Amersham (Arlington Heights, Ill.).

Neutrophil sequestration was measured by MPO activity determination using the O-dianisidine method as previously described (Barone, et al., 1991). Whole tissue protein (100 μg) extract was used to measure the MPO activity. Activity was expressed as the amount of enzyme necessary to generate a change in absorbance of 1.0 per min per mg wet weight of tissue.

Tissue protein (100 μg) was placed in 96-well plates. Expression of IL-1β and IL-6, TNFα, and p65 NF-κB protein in whole tissue lysates was determined using anti-mouse cytokine ELISA kits from Pierce Biotechnology, Inc. (Rockford, Ill.) as previously described (Slogoff, et al., 2004). Cytokine expression of MIP-2 was determined with an anti-mouse MIP-2 ELISA purchased from Assay Designs, Inc. (Ann Arbor, Mich.). Each ELISA was performed according to the manufacturer's instructions.

Excised colons were evaluated for approximate length, for blood content, stool consistency, and general appearance of bowel wall. Histological examination was performed on three samples of the colon from each animal. The samples were fixed in 10% buffered formalin, dehydrated in ethanol, and then embedded in paraffin. Sections (4 μm) were then prepared and stained with hematoxylin and eosin.

### EXAMPLE 7

Attenuation of Mucosal Inflammation Associated with DSS-Induced Colitis with siRNA Treatment

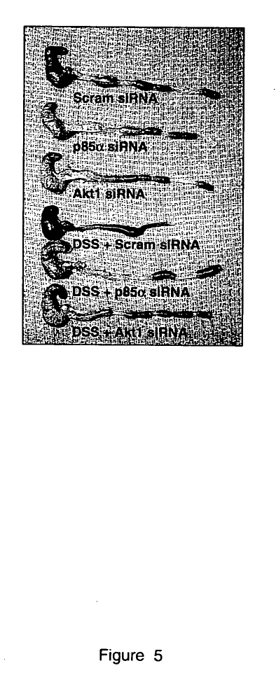

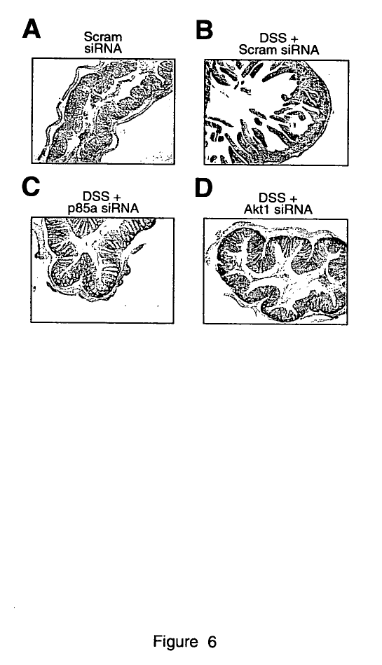

DSS treatment results in colonic shortening, intraluminal hemorrhage, and necrosis after prolonged exposure as previously described (Tessner, et al., 1998); treatment with either p85α or Akt1 siRNA abrogated the gross changes in the colon associated with DSS treatment (FIG. 5). siRNA to p85α appeared more effective than siRNA to Akt1 in preventing the bowel shortening, mucosal wall thinning and blood in the colon. Colonic sections were examined histologically with decreased leukocyte infiltration and mucosal damage noted with siRNA to either p85α or Akt1 compared with DSS-treatment and administration of control siRNA (FIG. 6, panels A-D).

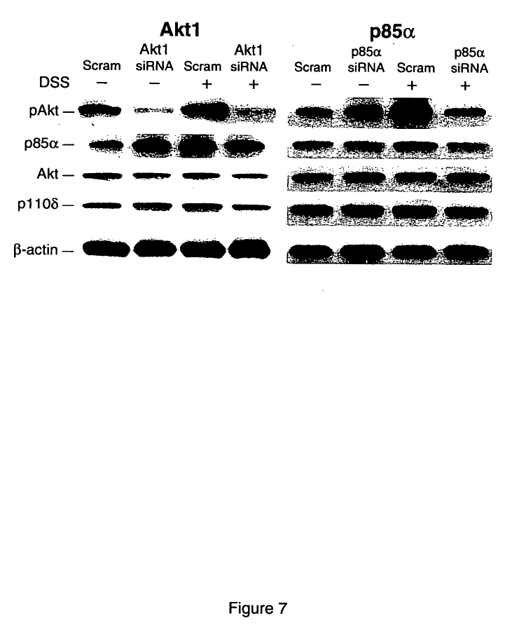

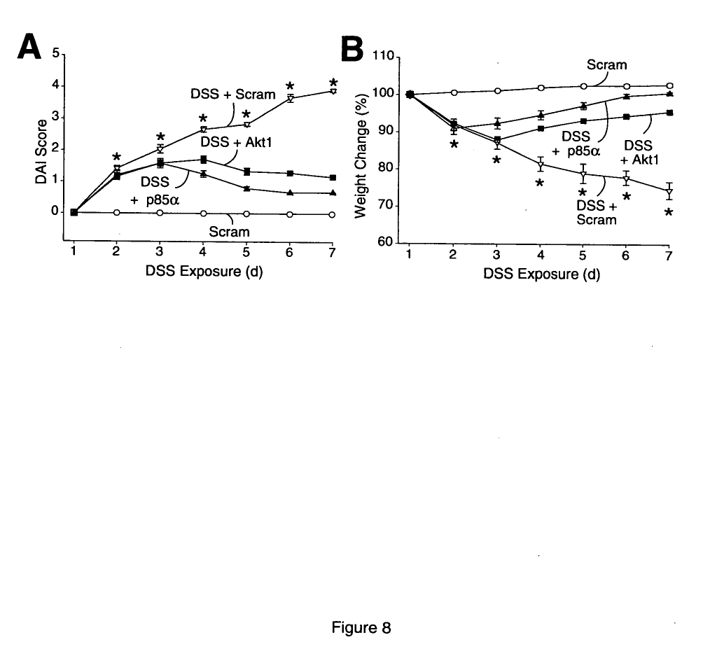

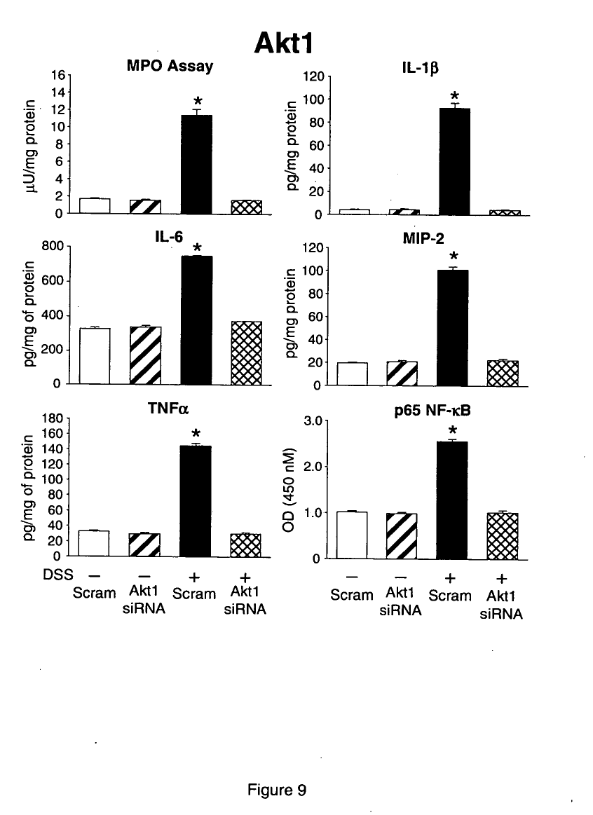

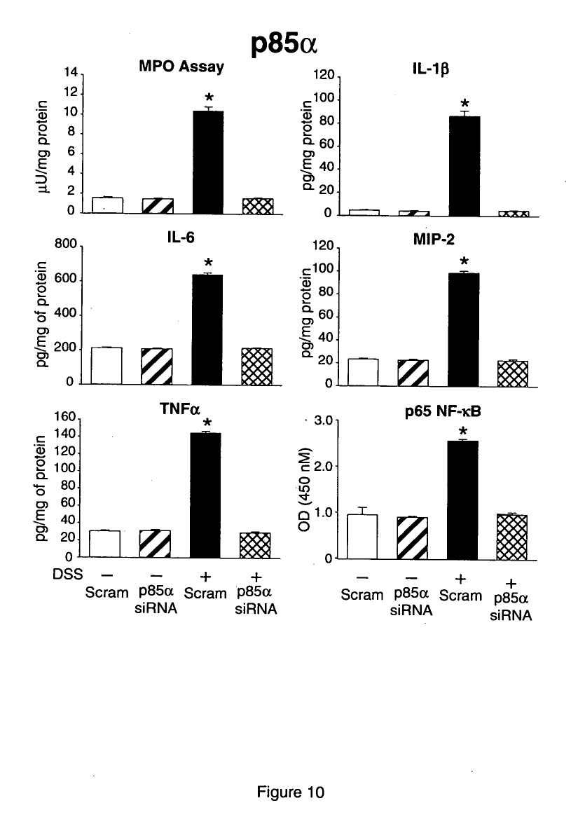

Akt1 siRNA treatment suppressed pAkt expression in the untreated colon and the induced pAkt expression noted after DSS treatment (FIG. 7, panel A). Expression of p85α and Akt was not significantly altered. In addition, expression of p110δ was assessed and found to be relatively unchanged with Akt1 siRNA. Treatment with siRNA directed to p85α decreased pAkt induction after DSS and decreased p85 expression in normal and DSS-treated colon; Akt and p110δ expression was not altered (FIG. 7, panel B). Quantitation of the disease activity index (DAI) and weight loss demonstrated an increase in DAI (FIG. 8, panel A) and a decrease in weight (FIG. 8, panel B) on days 1-3 in the DSS-treated mice; after day 3, treatment with either p85α or Akt1 siRNA stabilized and then improved both DAI and the weight loss with values returning to near control levels by day 7. In contrast, mice treated with DSS and control siRNA showed a progressive increase in DAI (˜4 on day 7) and decrease in weight (˜25% loss on day 7). Consistent with the DAI scores, treatment with either Akt1 siRNA (FIG. 9) or p85α siRNA (FIG. 10) significantly attenuated neutrophil sequestration, cytokine tissue levels, and p65 NF-κB activity in the colon compared to DSS+control siRNA treatment.

### EXAMPLE 8

Inhibition of the Inflammatory Changes Associated with TNBS-Induced Colitis by siRNA to p85α and Akt1

To determine whether siRNA directed p85α or Akt1 is effective in other models of colitis, TNBS (5 ng) was used to generate a colitis model. TNBS-induced colitis model demonstrated inflammatory changes resembling Crohn's disease. SiRNA was administered rectally on days 1 and 4 of a 7-day course.

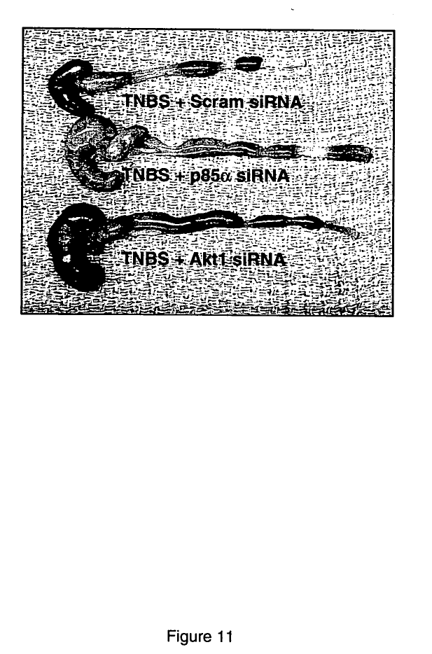

Similar to siRNA treatments in DSS-induced colitis in mice, siRNA treatments in TNBS-induced colitis in mice decreased the overall inflammatory effects of TNBS, including improved or normal bowel length and absent bowel wall hemorrhaging as shown in FIGS. 11-14.

### EXAMPLE 9

Cell Lines, Reagents and Antibodies

The human colorectal cancer cell lines KM20 and KM12C (derived from a Dukes' D or Dukes' B stage colon carcinoma, respectively) were obtained from Dr. Isaiah J. Fidler (M. D. Anderson Cancer Center, Houston, Tex.) (Morikawa, et al., 1988). KM20 and KM12C cells were grown in minimum Eagle's medium supplemented with 10% fetal bovine serum, 1% sodium pyruvate and 1% nonessential amino acids, 2% MEM Essential Vitamin and cultured at 37° C. under an atmosphere containing 5% CO₂. Tissue culture media and reagents were obtained from Life Technologies, Inc. (Grand Island, N.Y.). Rabbit polyclonal anti-caspase-3, mouse polyclonal anti-Bax and mouse polyclonal anti-PKBα, were purchased from BD Transduction Laboratories (San Jose, Calif.). Rabbit anti-phospho-Akt (Ser473), rabbit cleaved caspase-9 antibodies and rabbit Bcl-xL were purchased from Cell Signaling (Beverly, Mass.). Mouse monoclonal caspase-8 antibodies were purchased from Pharmingen (San Diego, Calif.). Mouse monoclonal anti-p85α antibody was purchased from Upstate (Charlottesville, Va.). Rabbit polyclonal anti-cytochrome C and rabbit polyclonal anti-PARP were obtained from Santa Cruz Biotechnology (Santa Cruz, Calif.). Mouse monoclonal anti-β-actin antibody was obtained from Sigma-Aldrich (St. Louis, Mo.). FITC-conjugated secondary antibodies were purchased from Molecular Probes (Eugene, Oreg.). Immobilon P membranes for Western blots were from Millipore Corp. (Bedford, Mass.), and X-ray film was purchased from Eastman Kodak (Rochester, N.Y.). The enhanced chemiluminescence (ECL) system for Western immunoblot analysis was from Amersham Biosciences (Arlington Heights, Ill.). Total RNA was isolated using TRIzol® Reagents (Invitrogen, Carlsbad, Calif.). The human apoptosis DNA template set (hAPO-2c and hAPO-2b) was from BD Pharmingen. [γ-³²P]ATP (3,000 Ci/mmol) was from Amersham Biosciences. siPORT™ Amine transfection agent and RPA-III kit were obtained from Ambion (Austin, Tex.). All other reagents were of molecular biology grade and purchased from Sigma-Aldrich.

### EXAMPLE 10

siRNA Synthesis and Transfection

Sequence information regarding human pik3r1 and Akt1 genes (GenBank NM_{—}019884 and NM_{—}005163) was extracted from the NCBI Entrez nucleotide database. Several siRNA with different targeting sequences for each gene were selected and each targeting segment was searched with NCBI Blast to confirm specificity only to the targeted gene. The pik3r1 specific siRNA targeted all three transcripts derived by alternative splicing of p85. The siRNA were synthesized using a transcription-based method with the Silencer siRNA construction kit according to the manufacturer's instructions. The 29-mer sense and antisense DNA oligonucleotide templates (21 nucleotides specific to the targets and 8 nucleotides specific to T7 promoter primer sequence 5′-CCTGTCTC-3′ (SEQ ID NO:7)) were synthesized (see Table 1) by Sigma-Genosys (Woodlands, Tex.). SiRNAs to p85α or Akt1 were synthesized targeting regions of Homo sapiens p85α or Akt1 and using the 29-mer DNA oligonucleotide templates (see Table 2). SiRNA to human pik3r1(3) have the same sense and antisense sequences as the siRNA to murine p85α, and siRNA to human Akt1(3) have the same sense and antisense sequences as the siRNA to murine Akt (see Above Example 4) due to the fact that the target sequences were chosen from part of the genome similar in human and mice. The purity of the synthesized siRNA was confirmed by agarose gel analysis.

Cell culture transfection was carried out with siPORT™ Amine transfection agent according to the manufacturer's protocol. Three days after transfection with the siRNA duplexes, cells were harvested and protein expression was assessed by Western blot. The templates for the synthetic siRNA molecules used in this study are shown in Table 1.

TABLE 1
Sequences of synthesized DNA oligonucleotide templates

| | | |
|---|---|---|
| Gene | Sense | Antisense |

| | | |
|---|---|---|
| pik3r1 | 5′- AAGCTAGGCGACCTAATAAGCCCTGTCTC -3′ | 5′- AAGCTTATTAGGTCGCCTAGCCCTGTCTC -3′ |
| (1) | (SEQ ID NO:8) | (SEQ ID NO:9) |
| pik3r1 | 5′- AAGGGATGTGCGGGTATATTCCCTGTCTC -3′ | 5′- AAGAATATACCCGCACATCCCCCTGTCTC -3′ |
| (2) | (SEQ ID NO:10) | (SEQ ID NO:11) |
| pik3r1 | 5′- AAGCTTTGTTTCGGTTGCTGCCCTGTCTC -3′ | 5′- AAGCAGCAACCGAAACAAAGCCCTGTCTC -3′ |
| (3) | (SEQ ID NO:12) | (SEQ ID NO:13) |
| Akt1 | 5′- AAGGCCACGATGACTTCCTTCCCTGTCTC -3′ | 5′- AAGAAGGAAGTCATCGTGGCCCCTGTCTC -3′ |
| (1) | (SEQ ID NO:14) | (SEQ ID NO:15) |
| Akt1 | 5′- AAGTGGGTCTGGAAAGAGTACCCTGTCTC -3′ | 5′- AAGTACTCTTTCCAGACCCACCCTGTCTC -3′ |
| (2) | (SEQ ID NO:16) | (SEQ ID NO:17) |
| Akt1 | 5′- AACACACTCCATGCTGTCATCCCTGTCTC -3′ | 5′- AAGATGACAGCATGGAGTGTGCCTGTCTC -3′ |
| (3) | (SEQ ID NO:18) | (SEQ ID NO:19) |

TABLE 2
Synthesized siRNA and targeting sequences

| | | Sense |
|---|---|---|
| | | vs. |
| Gene | Target | Antisense |
| pik3r1 | 5′- AAGCTTATTAGGTCGCCTAGC -3′ | 5′- GCUUAUUAGGUCGCCUAGCUU -3′vs. |
| (1) | (SEQ ID NO:20) | 3′- UUCGAAUAAUCCAGCGGAUCG -5′ |
| | | (SEQ ID NO:21 vs. SEQ ID NO:22) |
| pik3r1 | 5′- AAGAATATACCCGCACATCCC -3′ | 5′-zzzzzzGAAUAUACCCGCACAUCCCUU -3′vs. |
| (2) | (SEQ ID NO:23) | 3′-UUCUUAUAUGGGCGUGUAGGG -5′ |
| | | (SEQ ID NO:24 vs. SEQ ID NO:25) |
| pik3r1 | 5′- AAGCAGCAACCGAAACAAAGC -3′ | 5′-zzzzzzGCAGCAACCGAAACAAAGCUU -3′vs. |
| (3) | (SEQ ID NO:26) | 3′- UUCGUCGUUGGCUUUGUUUCG -5′ |
| | | (SEQ ID NO:2 vs. SEQ ID NO:3) |
| Akt1 | 5′- AAGAAGGAAGTCATCGTGGCC -3′ | 5′-zzzzzzGAAGGAAGUCAUCGUGGCCUU -3′vs. |
| (1) | (SEQ ID NO:27) | 3′-UUCUUCCUUCAGUAGCACCGG -5′ |
| | | (SEQ ID NO:28 vs. SEQ ID NO:29) |
| Akt1 | 5′- AAGTACTCTTTCCAGACCCAC -3′ | 5′-zzzzzzGUACUCUUUCCAGACCCACUU -3′vs. |
| (2) | (SEQ ID NO:30) | 3′-UUCAUGAGAAAGGUCUGGGUG -5′ |
| | | (SEQ ID NO:31 vs. SEQ ID NO:32) |
| Akt1 | 5′- AAGATGACAGCATGGAGTGTG -3′ | 5′-zzzzzzGAUGACAGCAUGGAGUGUGUU -3′vs. |
| (3) | (SEQ ID NO:33) | 3′- UUCUACUGUCGUACCUCACAC -5′ |
| | | (SEQ ID NO:5 vs. SEQ ID NO:6) |

### EXAMPLE 11

Protein Preparation and Western Immunoblot

Western immunoblot analyses were performed as described previously (Wang, et al., 2000). Cells were lysed with TNN buffer at 4° C. for 30 min. Lysates were clarified by centrifugation (10,000×g for 30 min at 4° C.) and protein concentrations determined using the method of Bradford (1976). Briefly, total protein (100 μg) was resolved on a 10% polyacrylamide gel and transferred to Immobilon-P nylon membranes. Filters were incubated overnight at 4° C. in blotting solution (Tris-buffered saline containing 5% nonfat dried milk and 0.1% Tween 20), followed by a 1-h incubation with primary antibodies. Filters were washed three times in a blocking solution and incubated with horseradish peroxidase-conjugated secondary antibodies for 1 h. After three additional washes, the immune complexes were visualized by ECL detection.

### EXAMPLE 12

Immunofluorescence Microscopy

KM20 cells were seeded onto sterile glass coverslips in 60 mm dishes and cultured for 16-24 h before treatment. Cells were transfected with pik3r1 or Akt1 or scrambled (control) siRNA sequences. At 96 h posttransfection, cells were washed in PBS, fixed in cold methanol for 5 min, and washed in three changes of PBS followed by a solution of 10% normal goat serum. After washing, cells were then incubated with the primary antibody (diluted in PBS with 1.5% normal goat serum) for 60 min. After washing with PBS, cells were incubated with FITC-conjugated secondary antibody (2 μg/ml) in PBS with 1.5% normal goat serum for 45 min. After three final washes, the slides were viewed with a fluorescence microscope.

### EXAMPLE 13

Ribonuclease (RNAse) Protection Assay

Cells were treated with pik3r1 and Akt1 siRNA and harvested 72 h later. RNA was isolated from cells using TRIzol® RNA reagent according to the manufacturer's protocol. A multiprobe template set (hAPO3c), which assesses expression of caspase 8, FASL, FAS, DR3, Decoy receptor 1 (DcR1), DR4, DR5, TRAIL, TNFR p55, TRADD and the housekeeping genes GAPDH and L32, as well as the Human Apoptosis hAPO-2b and hAPO-2c Template Set were labeled using Ambion T7 MAXIscript. The Ambion RPA III kit was then utilized to hybridize the RNA from the samples with the labeled probes. After RNAse treatment of hybridized samples, protected products were resolved on a 5% acrylamide gel, adsorbed onto filter paper, and exposed to Kodak BioMax MR film in a cassette at −70° C. and analyzed as previously described (Wang, et al., 2000).

### EXAMPLE 14

MTT Assay

Cells were transfected with pik3r1, Akt1 or scrambled siRNA at a density of 2×10⁵ cells/well in 12 well plates. After 24 h, adherent cells were detached by rapid trypsinization, counted in a Hausser chamber and replated into 96 well plates at a concentration of 2×10³ cells/well. Assays were performed at 72, 96 and 120 h post-transfection according to the manufacturer's protocol and as described (Iwase, et al., 1997).

### EXAMPLE 15

DNA Fragmentation Assay

Cells were transfected with pik3r1, Akt1 or scrambled siRNA. After 48 h, adherent cells were detached by rapid trypsinization, counted in a Hausser chamber and seeded at a density of 2×10³ cells per well in 96-well flat-bottom microtiter plates and incubated in 250 μl of growth medium for 24 h at 37° C. Media were removed and either new complete media or TRAIL (100 ng/mL) was added in a final volume of 200 μL and allowed to grow for 24 h at 37° C. After treatment, DNA fragmentation was evaluated by examination of cytoplasmic histone-associated DNA fragments (mono- and oligonucleosomes) using a Cell Death Detection ELISA^{Plus} kit (Roche Molecular Biochemicals) according to the manufacturer's instructions and as previously described (Wang. et al., 2002). The experiments were repeated on at least 3 separate occasions.

### EXAMPLE 16

In Vivo Experiments

For in vivo studies, 4-6 week old male nude^{nu/nu} mice were obtained from Harlan Sprague Dawley (Indianapolis, Ind.) and housed in clean pathogen-free rooms in an environment with controlled temperature (22° C.), humidity, and a 12 h light/dark cycle. The mice were fed standard chow (Formula Chow 5008; Purina Mills, St. Louis, Mo.) and tap water ad libitum and allowed to acclimate for one week.

Prior to tumor injection, KM20 cells were harvested, washed twice in ice-cold serum free minimum Eagle's medium, counted for viable cells by trypan blue exclusion. The cells were resuspended in the same medium and used to inject mice subcutaneously (sc) with 2×10⁶ cells in a volume of 0.2 ml. After tumors reached ˜60 mm², mice were sacrificed and 9 mm² tumor xenografts were transferred to the left flank of athymic mice. After tumor xenografts reached ˜60 mm² (day 10), mice were randomly divided into three groups (n=5 mice/group): the first group of mice was treated with scrambled siRNA, the second and third groups were treated with siRNA (5 μg diluted in 100 μl of serum free medium) directed against pik3r1 or Akt1, respectively, as intratumoral injections. Treatments consisted of electrotherapy initiated from 2 to 3 min after intratumoral siRNA injection once a day every 3 days. Tumor growth was assessed every third day from day 10 onward by measuring the two greatest perpendicular tumor dimensions using vernier calipers (Mitutozo, Tokyo, Japan) (Wang, et al., 2002). Consistent with institutional requirements, the mice were euthanized once tumors grew to a size greater that 2 cm.

### EXAMPLE 17

siRNA Electrotherapy

Electrotherapy consisted of intratumoral administration of pik3r1, Akt1 or scrambled control siRNA followed by exposure of tumor nodules to electric pulses. Square wave electric pulses of 70 μsec, 100 V amplitude were delivered through two parallel stainless steel electrodes (Tweezertrode Model 522, Holliston, Mass.) with an electropulsator BTX T 820 (Holliston, Mass.). In the study, plate electrodes with a distance of 10 mm between them were used for percutaneous delivery of electric pulses. Each run of electric pulses was delivered in two trains of tree pulses, with 1 sec interval, in two perpendicular directions. The orientation of the electric field was used to direct the uptake of the siRNA. Good contact between the electrodes and the skin was assured by means of a conductive gel. Nodules larger than 7 mm in diameter were treated with several runs of electric pulses, administered in adjacent positions in such a way that the whole tumor area was effectively treated.

### EXAMPLE 18

Statistical Analysis

Results are expressed as mean ±standard deviation (SD). All statistical analyses were performed on a personal computer with the SigmaStat v.2.03 software (SPCC Inc., San Rafael, Calif.). The data was analyzed using analysis of variance for a two-factor factorial experiment. The 2 factors were Akt1 (present and absent) and p85α (present and absent). All tests were assessed at the 0.05 level of significance.

### EXAMPLE 19

Suppression of pAkt Expression by pik3r1 or Akt1 siRNA

PI3K inhibition exhibits a potent antitumor effect in certain cancer cells including colorectal cancers (Wang, et al., 2002; Osaki, et al., 2004; and Murillo, et al., 2004); these effects appear to be due to inhibition of Akt/PKB phosphorylation (Itoh, et al., 2002). It is speculated that siRNA directed to PI3K/Akt pathway components would inhibit cell growth and induce apoptosis in human colorectal cancer cell lines. In order to minimize the potential for nonspecific effects in the application of siRNA in vitro and in vivo, effective siRNA for pik3r1 and Akt1 downregulation were identified (Table 1, Akt1(2) and pik3r1(2)) so that the lowest possible concentration of siRNA was used for gene silencing. In view of evidence that Akt plays a critical role in the prosurvival properties of PI3K (Nichol son, et al., 2002), activation of Akt in pik3r1 and Akt1 siRNA treated cells was then analyzed by Western blot and immunofluorescent analysis using a phospho-specific Ser473 Akt antibody.

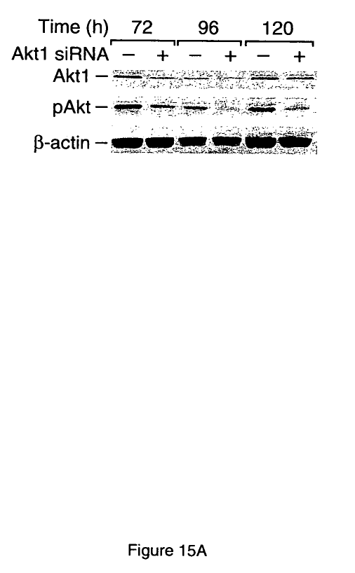

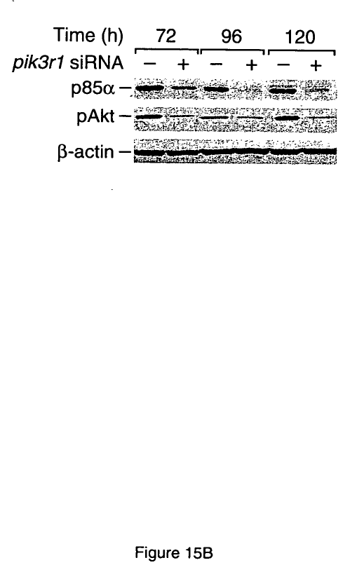

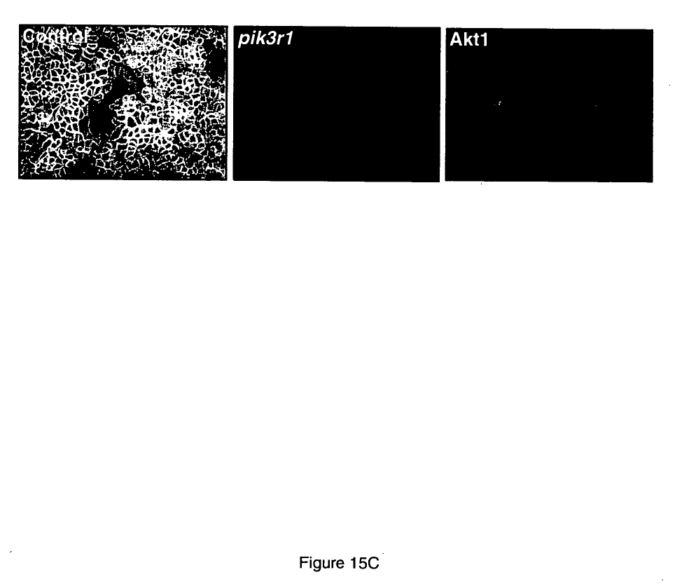

The results demonstrated that transfection with siRNA to either Akt1 (FIG. 15A) or pik3r1 (FIG. 15B) into KM20 cells reduced Akt1 and p85α protein levels, respectively, at 72, 96 and 120 h after transfection. Both Akt1 and pik3r1 siRNA suppressed pAkt expression levels. Consistent with the Western blot results, immunofluorescent analysis demonstrated suppression of pAkt expression in both pik3r1 and Akt1 siRNA transfected KM20 cells compared with scrambled sequences (control) (FIG. 15C). Similar results were noted in KM12C cells (data not shown).

### EXAMPLE 20

Reduction of Cell Survival and Induction of Colorectal Cancer Cell Apoptosis by pik3r1 and Akt1 siRNA

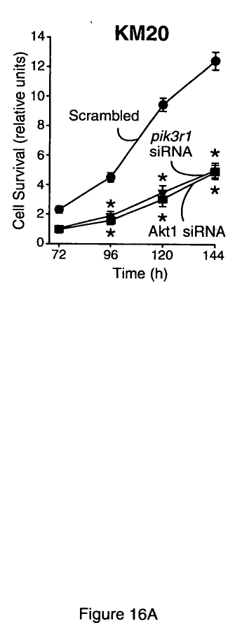

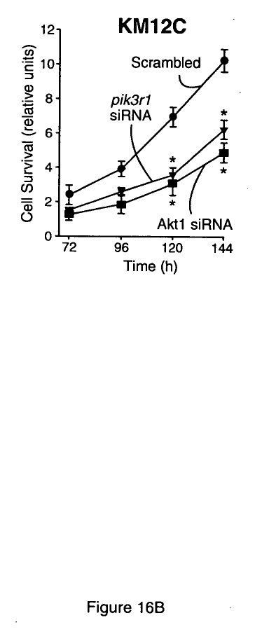

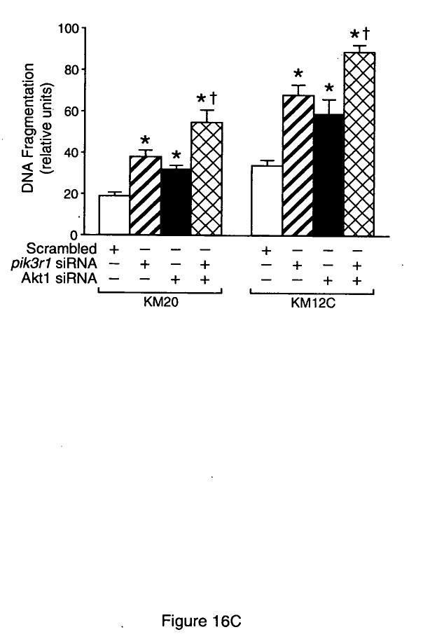

To determine the functional effects of pAkt suppression, the effect of siRNA treatment on the viability of KM20 and KM12C cells was examined by MTT assay. Transfection with either pik3r1 or Akt1 siRNA significantly decreased cell viability in KM20 (FIG. 16A) and KM12C (FIG. 166B) cells at 96, 120 and 144 h after transfection.

To determine whether this reduction in cell viability was a result of an induction of apoptosis, DNA fragmentation was measured by an ELISA method (FIG. 1 6C). An increase in DNA fragmentation, which is characteristic of apoptosis, was demonstrated in both KM20 and KM12C colorectal cancer cells with either pik3r1 or Akt1 siRNA compared to scrambled sequence (control). Moreover, the combination of pik3r1 and Akt1 siRNA resulted in increased apoptosis in both cell lines compared with either siRNA alone. These findings demonstrate that siRNA-mediated PI3K pathway suppression induces apoptotic cell death in both the KM20 and KM12C colorectal cancer cell lines.

### EXAMPLE 21

Caspase Activation, Inhibition of Anti-Apoptotic and Induction of Pro-Apoptotic Protein Expression

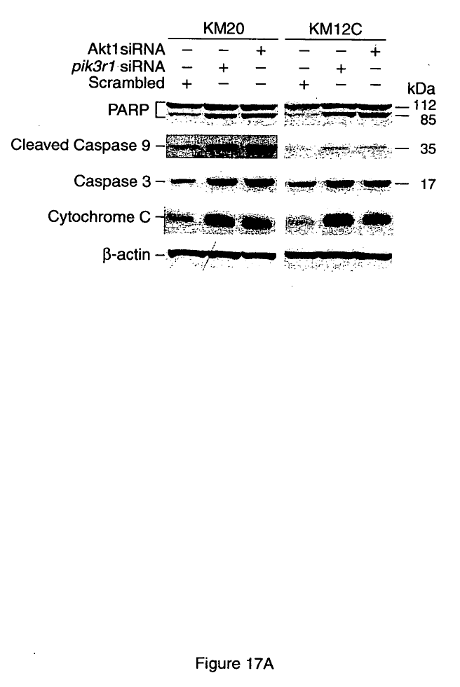

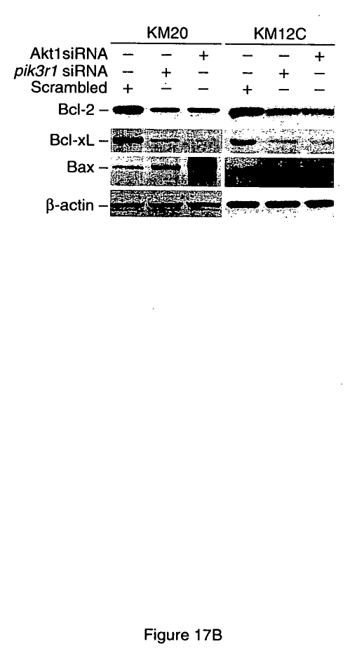

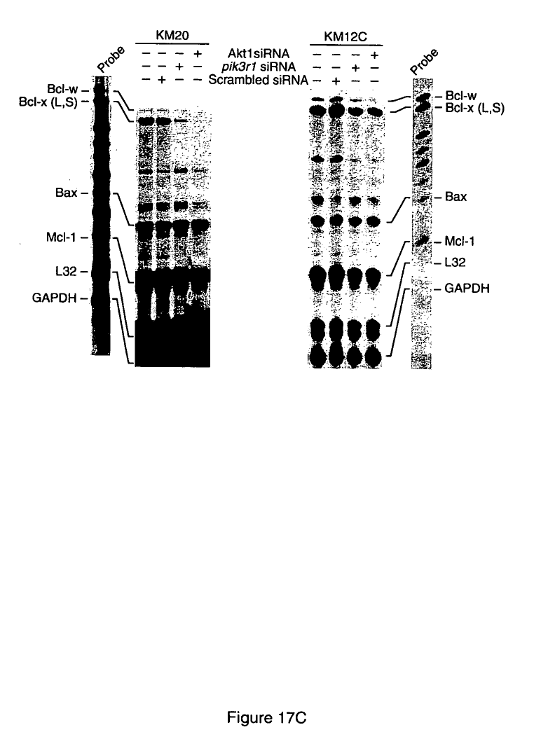

To further determine the molecular mechanisms involved in the induction of apoptosis in these colorectal cancer cells by pAkt suppression, the activation of the initiator caspase-9 and the effector caspase-3 was next determined (FIG. 17A). The released cytochrome C binds to Apaf-1 and induces a conformational change, which leads to the assembly of a heptamer Apaf-1/cytochrome C complex called the apoptosome (Green, et al., 1998). Within the apoptosome, each Apaf-1 subunit is bound noncovalently to a procaspase-9 subunit via their respective CARD domains, which results in the proteolytic cleavage and activation of caspase-9 (Salvesen, et al., 1997).

It is demonstrated that pik3r1 or Akt1 siRNA treatment induced cytochrome C release and resulted in caspase-9 activation, as shown by increased expression of its large active subunit (35 kDa), in both KM20 and KM12C cells (FIG. 17A). A similar analysis of caspase-3 demonstrated that siRNA to the PI3K/Akt components resulted in enhanced cleaved caspase-3 protein expression as noted by the 17 kDa cleavage product (i.e., active caspase-3). Increased caspase cleavage was associated with increased PARP cleavage, a downstream substrate of caspase-3, as noted by the 85 kDa cleavage product (Cai, et al., 1998). The blot was stripped and reprobed with 0-actin, demonstrating relatively equal protein loading. Taken together, these results demonstrate that caspase activation by PI3K/Akt siRNA treatment involves activation of caspase-9 and caspase-3 and release of cytochrome C in colorectal cancer cells, which suggests that apoptosis is mediated by the mitochondrial apoptotic pathway.

The mitochondrial pathway of caspase activation is regulated by the Bcl-2 family of proteins (Ruemmele, et al., 2003). Both Bcl-2 and Bcl-xL inhibit cytochrome C release and caspase-9 activation induced by a variety of cytotoxic agents (Cai, et al., 1998). Since a high percentage of colorectal cancers have increased levels of Bcl-2 or Bcl-xL (Kapiteijn, et al., 2001; Maurer, et al., 1998), the search for agents to overcome drug resistance in this cancer is an important consideration in chemoprevention. Expression of the antiapoptotic proteins Bcl-2 and Bcl-xL and the proapoptotic protein Bax was analyzed by Western blot (FIG. 17B). Transfection with either pik3r1 or Akt1 siRNA decreased expression levels of Bcl-2 and Bcl-xL proteins, whereas the expression of Bax was increased in both KM20 and KM12C cells. To determine if mRNA levels were altered in a similar fashion as protein levels, RNAe protection experiments were performed using a labeled multiprobe, which assesses expression of multiple genes of the Bcl-2 family (FIG. 17C). Transfection with Akt1 or pik3r1 siRNA decreased expression of Bcl-w and Bcl-x (L, S) in both KM20 and KM12C. In contrast, Bax and the antiapoptotic gene Mcl-1 exhibited minimal or no alteration in expression following transfection with the siRNA to the PI3K/Akt components. Expression of the constitutively expressed L32 and GAPDH genes confirmed equal loading.

### EXAMPLE 22

Inhibition of KM20 Tumor Growth in vivo by pik3r1 and Akt1 siRNA Treatment

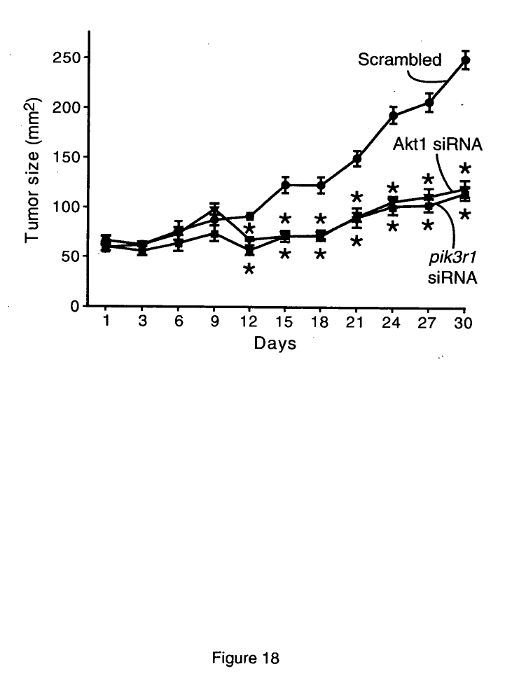

It was next determined whether intratumoral siRNA injection directed against pik3r1 or Akt1 followed by electroporation could alter the growth of KM20 tumor xenografts in nude mice (FIG. 18). An effective method to increase siRNA delivery into cells and tissues is by the local application of short, intense electric pulses (i.e., electropermeabilization). This approach is used in electrochemotherapy to potentiate the antitumor effectiveness of chemotherapeutic agents (Kuriyama, et al., 2000). KM20 colorectal cancer xenografts were transplanted into the flanks of athymic nude mice and then the mice were randomized to receive either Akt1, pik3r1 or scrambled (control) siRNA sequences. Treatment consisted of electrotherapy initiated 2-3 min after intratumoral siRNA injection every 3^{rd} day. In mice treated with scrambled sequence, the mean tumor volume increased >350% within 27 days after initiating treatment. Significant inhibitory effects on KM20 tumor growth were noted in the siRNA treatment groups (both Akt1 and pik3r1) by day 12. This inhibition persisted for the entire study period (total of 30 days).

### EXAMPLE 23

Reduction of Anti-Apoptotic Protein Expression by in vivo Treatment with Akt1 or pik3r1 siRNA

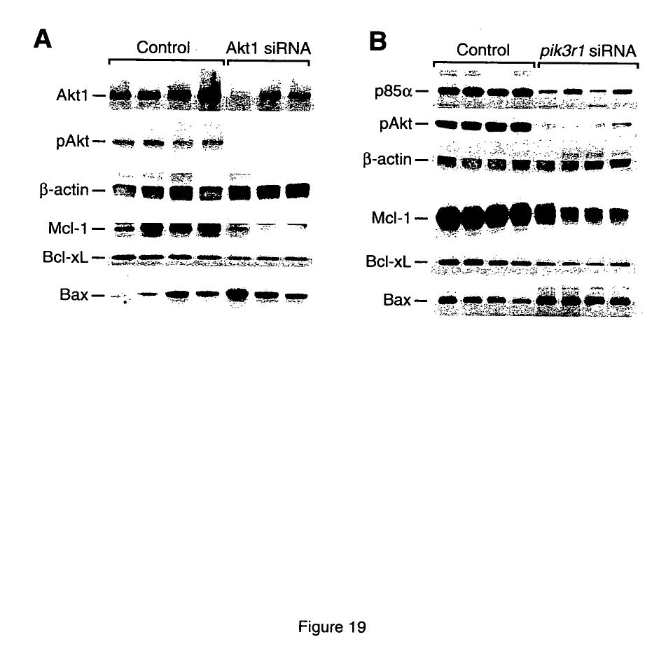

To determine next whether siRNA treatment to PI3K components in vivo can alter expression of apoptotic-related proteins, tumor xenografts were extracted for protein and Western blots performed (FIG. 19). Treatment with Akt1 siRNA decreased expression of the anti-apoptotic proteins Mcl-1 and Bcl-xL compared to scrambled sequence (control) (panel A). In contrast, expression of the proapoptotic protein, Bax, was increased with Akt1 siRNA. Suppression of both Akt1 and pAkt expression was noted with Akt1 siRNA treatment, whereas expression of β-actin was not affected. Similar to findings using Akt1 siRNA, treatment with pik3r1 siRNA decreased expression of Mcl-1 and Bcl-xL, but increased Bax expression (panel B). Furthermore, the effectiveness of the pik3r1 siRNA was confirmed by decreased p85α and pAkt expression. Taken together, these results in vivo are similar to in vitro findings using siRNA to either Akt1 or pik3r1. Moreover, the induction of Bax expression is consistent with the notion that pik3r1/Akt1 siRNA-induced apoptosis proceeds, in large part, through induction of mitochondrial injury and activation of the intrinsic apoptotic cascade.

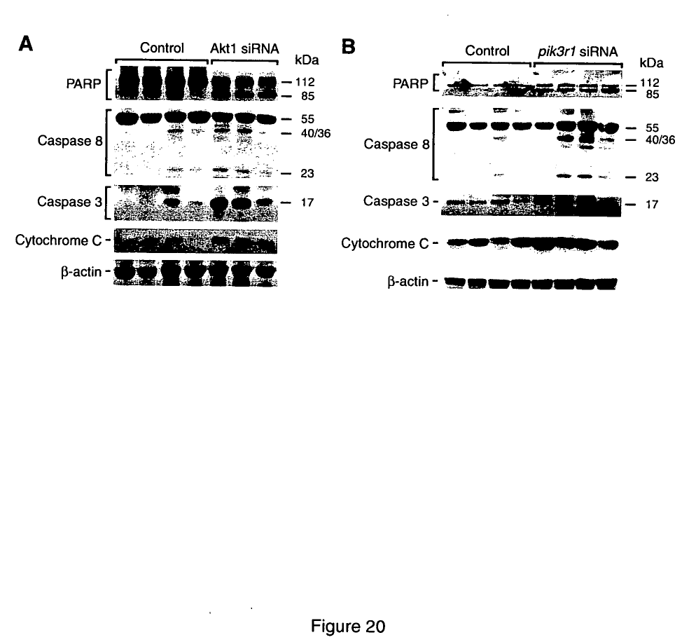

In vivo treatment with either Akt1 or pik3r1 siRNA activates the classic apoptotic pathway in KM20 tumor xenografts. In addition to assessment of the expression of the Bcl-2 family, activation of the caspase apoptotic pathway was also observed (FIG. 20). Similar to in vitro results, treatment with Akt1 siRNA (panel A) and pik3r1 siRNA (panel B) increased PARP cleavage and activation of caspase-8, -9 and -3. Likewise, increased cytochrome C expression was noted compared with scrambled control. Together, these results suggest that the suppression of PI3K/Akt decreased tumor growth through the activation of the classical caspase apoptotic pathway.

### EXAMPLE 24

Induction of DR4 and DR5 Expression by PI3K Inhibition

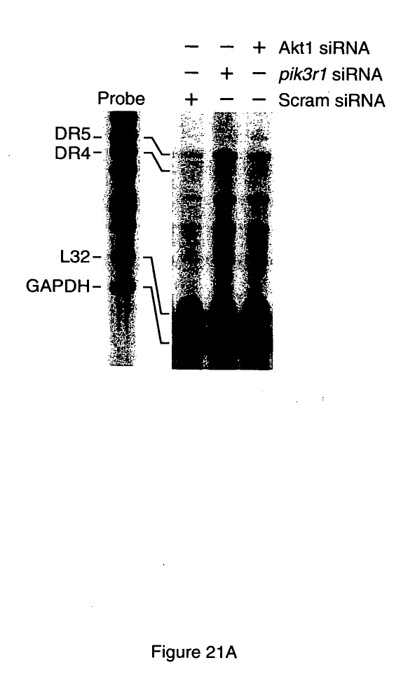

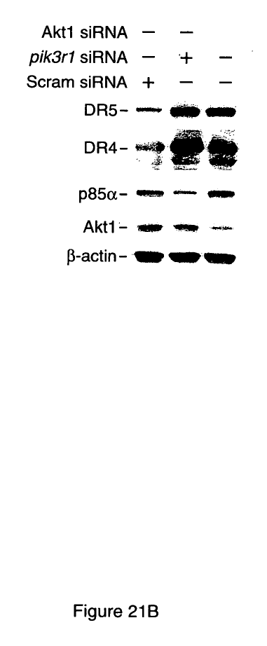

The PI3K/Akt pathway has been shown to protect cells from apoptosis and promote survival (Datta, et al., 1997). Treatment with the PI3K inhibitor wortmannin has been previously shown to induce TNF-related apoptosis-inducing ligand (TRAIL) mRNA expression in HT29 colorectal cancer cells (Wang, et al., 2002). To evaluate whether PI3K/Akt inhibition affects expression of genes and proteins that contribute to the apoptotic pathway in cells, KM20 cells were transfected with pik3r1 siRNA, Akt1 siRNA or the scrambled (scram) control and RNAe protection analysis, using a multiprobe template, or Western blot analysis was performed (FIGS. 21A and 21B). The results demonstrate that treatment with pik3r1 or Akt1 siRNA, but not scrambled siRNA, increased mRNA expression of DR4 and DR5 with no marked change in the expression of the other apoptotic-related genes contained in this probe set (FIG. 21A). Western blot analysis of similarly treated KM20 cells showed increased protein expression for DR4/DR5, consistent with the increases in mRNA levels (FIG. 21B). Specificity of pik3r1 or Akt1 siRNA treatment was confirmed by decreased expression of phosphorylated Akt (pAkt), the active form of Akt, and p85α and Akt1 protein levels. Equal β-actin expression indicates equal loading. Taken together, these data suggest that the PI3K/Akt pathway negatively regulates DR4/DR5 expression and is consistent with the role of PI3K/Akt as a cell survival pathway in colorectal carcinoma.

### EXAMPLE 25

PI3K Inhibition Augments TRAIL-Mediated Apoptosis

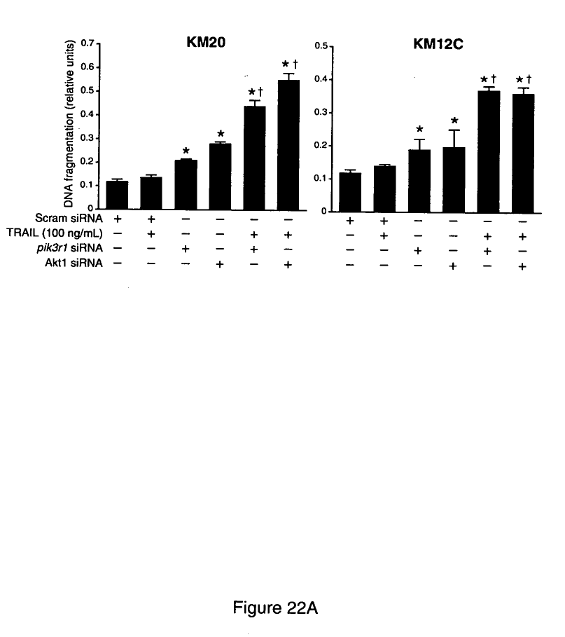

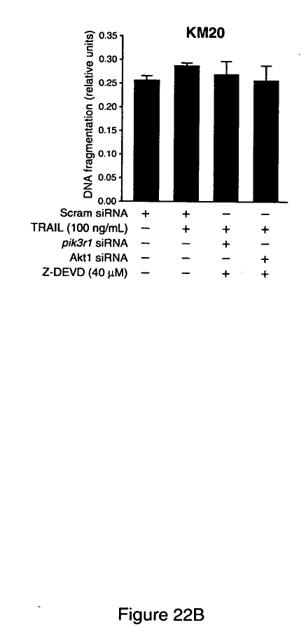

In order to further define the regulation of TRAIL-induced apoptosis, colorectal cancer cells were transfected with siRNA to either, pik3r1 or Akt1, and then treated with TRAIL (100 ng/mL) or vehicle. Apoptosis was assessed by quantitation of DNA fragmentation, a hallmark of apoptosis (Wyllie, et al., 1980) (FIGS. 22A and 22B). The results demonstrate that pik3r1 or Akt1 siRNA induced apoptosis in KM20 and KM12C cells, and, as previously shown, TRAIL alone has no effect on KM20 or KM12C cell growth (Hernandez, et al., 2001; Hernandez, et al., 2001) (FIG. 22A). In contrast, a significant augmentation in TRAIL-mediated apoptosis was observed with the combination of pik3r1 or Akt1 siRNA. These data suggest that upregulation of DR4 and DR5 expression by PI3K inhibition contributes to increased TRAIL-mediated apoptosis.

It was next examined whether caspase-3 activation was necessary for the increased apoptosis mediated by PI3K inhibition. KM20 cells were pretreated with either the caspase-3 inhibitor (Z-DEVD-fmk) or vehicle prior to treatment with TRAIL (100 ng/mL) and either pik3r1 or Akt1 siRNA (FIG. 22B). Z-DEVD-fmk completely blocked the induction of apoptosis mediated by the combination of TRAIL and pik3r1 or Akt1 siRNA. These data corroborate the hypothesis that reduction in pAkt expression and upregulation of DR4/DR5 receptor expression facilitate TRAIL-mediated caspase-3 activation and subsequent apoptosis.

### EXAMPLE 26

Characterization of TRAIL-Induced Apoptosis after siRNA Treatment

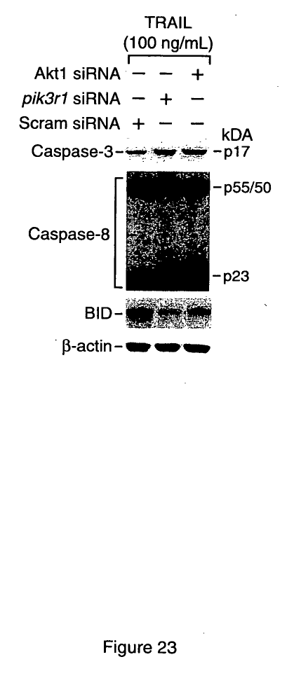

Caspases are responsible for most of the morphological and biochemical changes observed in apoptosis (Zheng, et al., 1998). In the extrinsic pathway, ligand binding and oligomerization of the TNF surface receptors lead to the activation of caspase-8 and/or caspase-10. In contrast, the intrinsic pathway is initiated by changes in the permeability of the mitochondrial membrane resulting in the release of cytochrome C and caspase-9 activation. The initiator caspases-8, -9, and -10 recruit the effector caspases-3, -6, and -7, which facilitate the remainder of the apoptotic process by specific proteolysis. To further evaluate the mechanism of apoptosis induction after pik3r1 or Akt1 siRNA transfection and TRAIL treatment in KM20 cells, expression of BID, which compromises mitochondrial integrity via activation and oligomerization of BAX and BAK at the mitochondria; caspase-8, which cleaves and activates BID; and caspase-3 were analyzed by Western blot (FIG. 23). TRAIL, in combination with pik3r1 or Akt1 siRNA, activated caspase-8, as noted by its proteolytic processing and BID cleavage. A similar analysis of caspase-3 demonstrated enhanced cleavage of pro-caspase-3 with the combination of TRAIL and pik3r1 or Akt1 siRNA compared with TRAIL treatment alone. The blot was stripped and reprobed with β-actin, demonstrating relatively equal protein loading. Taken together, these results suggest that apoptosis induction is predominantly mediated through the extrinsic apoptotic pathway mediated by caspase-8 and caspase-3 activation.

### EXAMPLE 27

Experimental Murine Model for Severe Acute Pancreatitis

Choline and methionine deficient with ethionine (CMDE) was used to generate pancreatitis model in mice. Female Swiss-Webster mice were divided into two groups: control siRNA or p85α siRNA treatment group. Mice were injected with 10 μg control or p85α siRNA 3 days before and 1 day after starting experiment using hydrodynamic tail vein method. Mice were fed CMDE diet for 3 days. Ethionine (250 mg/kg, twice a day) was fed via gavage. After being fed with CMDE diet for 3 days, mice were fed with regular lab food. The mortality for 7 days was assessed in both groups.

### EXAMPLE 28

Experimental Results

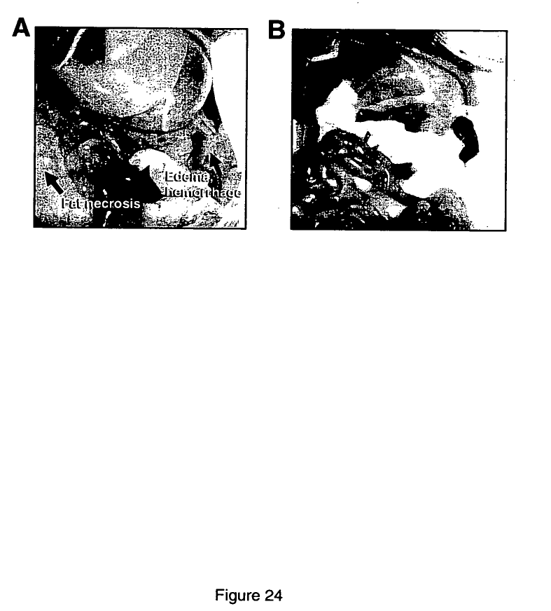

As shown in FIG. 24, control group mice with CMDE diet had moderate edema and hemorrhage in pancreas and fat necrosis in abdominal cavity (panel A). On the other hand, p85α siRNA injected mice with CMDE diet had no edema and bleeding in pancreas (panel B). These results suggest that p85α siRNA blocks the development of acute pancreatitis.

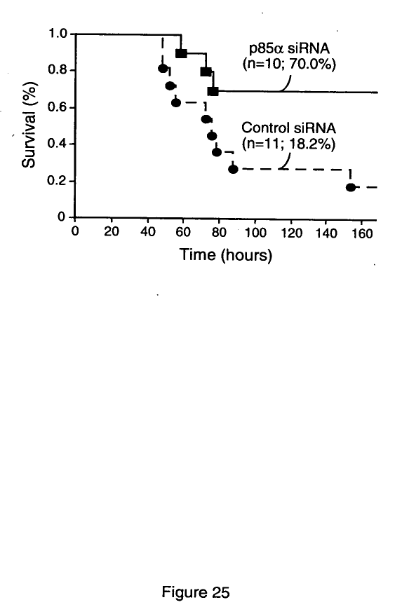

Result of mortality of control group mice vs. p85α siRNA treated mice with CMDE is shown in FIG. 25. Approximately 81.8% mice with control siRNA injection died after 7 days, whereas 30% mice with p85α siRNA injection died after the same period of time. These findings suggest that p85α siRNA reduces the development of acute pancreatitis and decreases the mortality.

### EXAMPLE 29

Conclusions and Discussions

The present study is the first to show that inhibition of PI3K isoforms by locally administered siRNA attenuates the mucosal inflammation in a murine model of inflammatory bowel disease (IBD). Local instillation of siRNA targeting specific signaling pathways (e.g., PI3K) represents a novel strategy to treat the inflammation associated with IBD.

Previous studies in experimental models of colitis in mice have used oral or systemic administration to deliver medication or treatments to affect changes in the inflammatory state. Currently available schemes to treat the inflammation in experimental models of colitis as well as actual patient cases of colitis work through non-specific inhibitors of inflammatory cascade propagation, which may carry undue risk from potentially harmful side effects and are not effective in treating the inflammation. In contrast, the present study uses localized delivery of specific and selective inhibitors of inflammatory cascade propagation, e.g., siRNA, to target tissues within the colon to assess changes to signal pathways within cells. New siRNA will be designed with specific sequences targeting inflammatory cascade propagation. Additionally, it is believed that other types of nucleic acid inhibitors such as anti-sense nucleic acid, decoy RNA, dsRNA and aptamers can also be designed to target specific components of inflammatory cascade propagation and delivered to diseased tissues that are in need of a therapy. The method of delivering the nucleic acid inhibitors can also be topical, oral and systemic.

There are number of diseases of the colonic mucosa such as colorectal cancers, inflammatory bowel disease, colonic dysplasia, and enterocolitis. Current medical procedures allow for easy access to the colonic mucosa of patients using conventional colonoscopy. Therapy using colonoscopy is limited to polypectomies, mucosal biopsies, and steroid bathing of the colonic mucosa. Since siRNA therapy requires only direct infusion of the siRNA solution to mucosal lesions, directed colonoscopy to treat mucosal diseases is possible and bound only by the limitations of the procedure of colonoscopy itself. The direct siRNA therapy described herein may be expanded and practiced by clinicians for treating patients with colonic mucosal diseases using siRNA directed against specific cellular markers administered locally via endoscopy of the colon.

The present in vivo data suggest that siRNA may have therapeutic potential for inhibiting the expression of genes that enhance the growth of tumors. Transfection of siRNA directed against pik3r1 or Akt1 led to reduced proliferation of KM20 colorectal cancer xenografts in nude mice. The ability of these siRNA to suppress pik3r1 and Akt1 levels and reduce the in vivo growth of KM20 tumor xenografts suggests that targeted therapy utilizing siRNA may represent a novel treatment strategy. A variety of other oncogenic and mutant tumor suppressor genes, such as Ras, Bcl-2, MDR, and specific components of the NF-κB pathway that are overexpressed in cancer cells may also be targeted by siRNA in an attempt to reduce their levels and thus decrease the proliferation of tumor cells.

Improving the efficacy of chemotherapy and radiotherapy in cancer represents another potential application of siRNA-based technology. Acquired resistance to anti-tumour drugs is encountered in approximately one-third of all cancer patients undergoing chemotherapy (Jimenez, et al., 1998). Various mechanisms have been proposed to explain the development of drug resistance in tumor cells, including impaired drug transport inside and from within the cell and increased anti-apoptotic potential of tumor cells (Jimenez, et al., 1998). One form of multidrug resistance is caused by overexpression of the MDR gene product, leading to a reduced intracellular concentration of the drug. Inhibition of MDR gene expression by siRNA has been shown to enhance the intracellular accumulation of various chemotherapeutic drugs and selectively restore chemosensitivity (Nieth, et al., 2003). Two important obstacles must be overcome for this potential to be realized. First, drug delivery techniques must be refined to provide more specific uptake in cancer cells. In the present study, electrotherapy consisting of intratumoral administration of pik3r1 or Akt1 siRNA followed by exposure of tumor nodules to percutaneous delivery of electric pulses was utilized to deliver the siRNA in vivo. Second, the ability to modify RNA oligonucleotides so that they are more stable in vivo will be necessary before adopting this technique for in vivo therapy. In this regard, proprietary chemical modifications have been developed that dramatically enhance both the stability and silencing longevity of siRNA while improving its potency and decreasing cellular toxicity (Khaleghpour, et al., 2004; Insinga, et al., 2005). These modifications now enable studies that were previously not feasible due to instability of the siRNA duplex or short duration of siRNA-mediated silencing and may provide for agents that are more clinically applicable for treating disease states that require longer acting effects.

siRNA used in the present study demonstrate that targeted suppression of pik3r1 or Akt1 levels can reduce colorectal cancer growth and enhance apoptosis both in vitro and in vivo. Targeted inhibition of PI3K pathway components may prove useful in the treatment of colorectal cancers either to increase tumor cell death or, more likely, to enhance the sensitivity of chemoresistant cancers to the effects of other chemotherapeutic agents. In fact, inhibition of PI3K/Akt in the present study is found to enhance the sensitivity of colorectal cancers to the apoptotic effects of TNF-related apoptosis-inducing ligand (TRAIL). This suggests that the targeted inhibition of the PI3K/Akt pathway may provide a novel method to enhance the sensitivity of chemoresistant colorectal cancers and, represent a useful adjuvant therapy in the treatment of these cancers. Given its specificity and the lower concentrations needed to inhibit gene expression, as compared with those required for antisense oligonucleotides, siRNA may have potential therapeutic utility in a variety of disease states, including certain other cancers.

The concerns regarding TRAIL resistance in certain tumors have prompted investigators to identify potential strategies to enhance the tumoricidal activity of TRAIL treatment. Both functional and decoy receptors in TRAIL-resistant cancer cell lines have previously been identified (Hernandez, et al., 2001; Wang, et al., 2002; Pan, et al., 1997; Kim, et al., 2000), however, the decoy hypothesis cannot entirely explain the resistance of these cells to the apoptotic effects of TRAIL treatment. It has been shown that inhibition of NF-κB, which controls the transcription of genes fundamental for survival, sensitizes colorectal cancer cells to TRAIL treatment. For example, agents that specifically target NF-κB, or the downstream targets of NF-κB activation (e.g., FLIP), may be useful to enhance the effectiveness of chemotherapeutic agents in certain cancers (Thomas, et al., 2002). Growth factors such as epidermal growth factor or insulin-like growth factor-1 also inhibit TRAIL-induced apoptosis through the Akt pathway. Akt exerts its antiapoptotic function by its ability to phosphorylate components of the cellular apoptotic regulatory circuit, such as BAD, Forkhead transcription factors, and by activating NF-κB. Akt phosphorylates and activates IKK-α, which in turn phosphorylates I-κB and promotes the nuclear translocation of NF-κB.

In the present study, the Akt/PKB signaling pathway has been shown to play an essential role in protecting cancer cells from the effects of apoptotic-inducing agents such as TRAIL. The role of activated Akt in the chemotherapeutic resistance of cancers to apoptosis has been reported which further support a prosurvival function for Akt (Pan, et al., 1997; Kandasamy, et al., 2002). Signaling of growth factors translocates Akt to the inner surface of the plasma membrane in proximity to regulatory kinases that phosphorylate and activate Akt. Human colorectal cancer cell lines (KM12C, KML4A, and KM20) were previously shown to be resistant to the effects of TRAIL treatment (Hernandez, et al., 2001). Because PI3K targets Akt for survival, the activation of Akt is modulated in the present study using a genetic approach to downregulate active Akt by transfecting KM20 or KM12C cells with pik3r1 or Akt1. Downregulation of Akt by pik3r1 or Akt1 transfection rendered KM20 and KM12C cells susceptible to TRAIL-induced apoptosis.

Other types of inflammation such as pancreatic inflammation can also be treated with siRNA targeting PI3K/Akt pathway. SiRNA directed against p85α subunit of the pathway is shown in the present study to block pancreatic inflammation and increase survival in the severe acute pancreatitis mouse model. Other types of inflammation having other causes when associated with bacterial, parasitic and viral infections can also be treated using the siRNA therapy. Examples include sepsis, cardiovascular and myocardial, kidney, dermatological (e.g., psoriasis), pulmonary (e.g., allergic airway inflammation), and neurological pathologies, chronic immuno-inflammatory diseases such as rheumatoid arthritis, multiple sclerosis, atherosclerosis. Further more, chronic inflammation caused by non-infectious agents such as smoke, asbestosis, coal, silica dust can also be treated using the siRNA therapy.

A pharmaceutical composition may be constructed comprising isolated and purified siRNA targeting the phosphoinositide 3-kinase (PI3K) signal transduction pathway and a pharmaceutically acceptable carrier. Further more, a system of administering siRNA-based therapy to an in-need individual might also be designed. In this system, isolated and purified siRNA are administered to the individual suffering from an inflammatory disease via intraperitoneal (IP) injection and rectal enema.

While the invention has been shown in only a few of its forms, it should be apparent to those skilled in the art that it is not so limited but susceptible to various changes without departing from the scope of the invention.

1. Small interfering RNA (siRNA) targeting a phosphoinositide 3-kinase (PI3K) signal transduction pathway.

2. The siRNA of claim 1, wherein the siRNA comprises double-stranded siRNA directed against p85α of the PI3K signal transduction pathway, wherein a first strand of the double-stranded siRNA comprises a sequence selected from the group consisting of SEQ ID NO:2, SEQ ID NO:21, and SEQ ID NO:24, and wherein a second strand of the double-stranded siRNA comprises a sequence selected from the group consisting of SEQ ID NO:3, SEQ ID NO:22, and SEQ ID NO:25.

3. The siRNA of claim 1, wherein the siRNA comprises double-stranded siRNA directed against Akt of the PI3K signal transduction pathway, wherein a first strand of the double-stranded siRNA comprises a sequence selected from the group consisting of SEQ ID NO:5, SEQ ID NO:28, and SEQ ID NO:31, and wherein a second strand of the double-stranded siRNA comprises a sequence selected from the group consisting of SEQ ID NO:6, SEQ ID NO:29, and SEQ ID NO:32.

4. A method for treating an individual suffering from an inflammatory disease, comprising the step of:
administering to the individual with a selective inhibitor targeting a specific component of inflammatory cascade propagation through a localized delivery.

5. The method of claim 4, wherein the selective inhibitor comprises small interfering RNA (siRNA).

6. The method of claim 5, wherein the inflammatory cascade propagation comprises a phosphoinositide 3-kinase (Pl3K) signal transduction pathway, and wherein the selective inhibitor comprises siRNA directed against components of the PI3K signal transduction pathway.

7. The method of claim 6, wherein the selective inhibitor comprises double-stranded siRNA directed against p85w of the PI3K signal transduction pathway, wherein a first strand of the double-stranded siRNA comprises a sequence selected from the group consisting of SEQ ID NO:2, SEQ ID NO:21, and SEQ ID NO:24, and wherein a second strand of the double-stranded siRNA comprises a sequence selected from the group consisting of SEQ ID NO:3, SEQ ID NO:22, and SEQ ID NO:25.

8. The method of claim 6, wherein the selective inhibitor comprises double-stranded siRNA directed against Akt of the PI3K signal transduction pathway, wherein a first strand of the double-stranded siRNA comprises a sequence selected from the group consisting of SEQ ID NO:5, SEQ ID NO:28, and SEQ ID NO:31, and wherein a second strand of the double-stranded siRNA comprises a sequence selected from the group consisting of SEQ ID NO:6, SEQ ID NO:29, and SEQ ID NO:32.

9. The method of claim 4, wherein the inflammatory disease is selected from the group consisting of a cancer, inflammatory bowel disease, pancreatitis, colonic dysplasia, enterocolitis, arthritis, an autoimmune disease, a chronic inflammatory state associated with organ transplantations, and a chronic inflammatory state associated with infection, a toxin or allergy.

10. The method of claim 9, wherein the cancer is a colorectal cancer.

11. The method of claim 4, wherein the step of administering comprises the step of:
directly delivering the selective inhibitor to a diseased tissue of the individual.

12. The method of claim 11, further comprising the step of:
exposing the diseased tissue to electric pulses.

13. A method for treating an individual suffering from an inflammatory disease of colonic mucosa, comprising the step of:
administering to the individual with a selective inhibitor targeting a phosphoinositide 3-kinase (PI3K) signal transduction pathway through a localized delivery.

14. The method of claim 13, wherein the selective inhibitor comprises small interfering RNA (siRNA).

15. The method of claim 14, wherein the selective inhibitor comprises double-stranded siRNA directed against p85α of the PI3K signal transduction pathway, wherein a first strand of the double-stranded siRNA comprises a sequence selected from the group consisting of SEQ ID NO:2, SEQ ID NO:21, and SEQ ID NO:24, and wherein a second strand of the double-stranded siRNA comprises a sequence selected from the group consisting of SEQ ID NO:3, SEQ ID NO:22, and SEQ ID NO:25.

16. The method of claim 14, wherein the selective inhibitor comprises double-stranded siRNA directed against Akt of the PI3K signal transduction pathway, wherein a first strand of the double-stranded siRNA comprises a sequence selected from the group consisting of SEQ ID NO:5, SEQ ID NO:28, and SEQ ID NO:31, and wherein a second strand of the double-stranded siRNA comprises a sequence selected from the group consisting of SEQ ID NO:6, SEQ ID NO:29, and SEQ ID NO:32.

17. The method of claim 13, wherein the inflammatory disease of colonic mucosa is selected from the group consisting of a colorectal cancer, inflammatory bowel disease, colonic dysplasia, and enterocolitis.

18. The method of claim 13, wherein the step of administering comprises the step of:
directly delivering the selective inhibitor to a diseased tissue of the individual through a rectal enema.

19. The method of claim 18, further comprising the step of:
exposing the diseased tissue to electric pulses.

20. A method for treating an individual having a cancer, comprising the step of:
co-administering to the individual with a chemotherapeutic drug and a selective inhibitor targeting a phosphoinositide 3-kinase (PI3K) signal transduction pathway.

21. The method of claim 20, wherein the selective inhibitor comprises double-stranded siRNA directed against p85α of the PI3K signal transduction pathway, wherein a first strand of the double-stranded siRNA comprises a sequence selected from the group consisting of SEQ ID NO:2, SEQ ID NO:21, and SEQ ID NO:24, and wherein a second strand of the double-stranded siRNA comprises a sequence selected from the group consisting of SEQ ID NO:3, SEQ ID NO:22, and SEQ ID NO:25.

22. The method of claim 20, wherein the selective inhibitor comprises double-stranded siRNA directed against Akt of the PI3K signal transduction pathway, wherein a first strand of the double-stranded siRNA comprises a sequence selected from the group consisting of SEQ ID NO:5, SEQ ID NO:28, and SEQ ID NO:31, and wherein a second strand of the double-stranded siRNA comprises a sequence selected from the group consisting of SEQ ID NO:6, SEQ ID NO:29, and SEQ ID NO:32.

23. The method of claim 20, wherein the cancer is a chemoresistant cancer, and wherein the chemotherapeutic drug is selected from the group consisting of an apoptosis-inducing agent, a NF-κB inhibitor, a growth factor inhibitor, a cell proliferation inhibitor, and an inhibitor of MDR gene expression.

24. The method of claim 23, wherein the apoptosis-inducing agent is a TNF-related apoptosis inducing ligand (TRAIL).

25. A method for increasing efficacy of chemotherapy in a cancer treatment, comprising the step of:
co-administering to an individual in need of the cancer treatment with a chemotherapeutic drug and a selective inhibitor targeting a phosphoinositide 3-kinase (PI3K) signal transduction pathway.

26. The method of claim 25, wherein the selective inhibitor comprises double-stranded siRNA directed against p85α of the PI3K signal transduction pathway, wherein a first strand of the double-stranded siRNA comprises a sequence selected from the group consisting of SEQ ID NO:2, SEQ ID NO:21, and SEQ ID NO:24, and wherein a second strand of the double-stranded siRNA comprises a sequence selected from the group consisting of SEQ ID NO:3, SEQ ID NO:22, and SEQ ID NO:25.

27. The method of claim 25, wherein the selective inhibitor comprises double-stranded siRNA directed against Akt of the PI3K signal transduction pathway, wherein a first strand of the double-stranded siRNA comprises a sequence selected from the group consisting of SEQ ID NO:5, SEQ ID NO:28, and SEQ ID NO:31, and wherein a second strand of the double-stranded siRNA comprises a sequence selected from the group consisting of SEQ ID NO:6, SEQ ID NO:29, and SEQ ID NO:32.

28. The method of claim 25, wherein the chemotherapeutic drug is selected from the group consisting of an apoptosis-inducing agent, a NF-κB inhibitor, a growth factor inhibitor, a cell proliferation inhibitor, and an inhibitor of MDR gene expression.

29. The method of claim 28, wherein the apoptosis-inducing agent is a TNF-related apoptosis inducing ligand (TRAIL).

30. A pharmaceutical composition comprising siRNA targeting a phosphoinositide 3-kinase (PI3K) signal transduction pathway and a pharmaceutically acceptable carrier.
